(19) United States
(12) Patent Application Publication
(10) Pub. No.: US 2010/0047892 A1
(43) Pub. Date: Feb. 25, 2010

(54) METHOD FOR MODIFYING CELLS

(75) Inventors: Jarmo Laine, Helsinki (FI); Tero Satomaa, Helsinki (FI); Jari Natunen, Vantaa (FI); Taina Jaatinen, Helsinki (FI); Annamari Heiskanen, Helsinki (FI); Johanna Nystedt, Helsinki (FI)

Correspondence Address: BIRCH STEWART KOLASCH & BIRCH, PO BOX 747, FALLS CHURCH, VA, 22040-0747, US

(73) Assignees: SUOMEN PUNAINEN RISTI,VERIPALVELU, Helsinki (FI); Glykos Finland Ltd., Helsinki (FI)

(21) Appl. No.: 12/522,853

(22) PCT Filed: Jan. 18, 2008
(86) PCT No.: PCT/FI2008/050015

§ 371 Date: Nov. 5, 2009

(30) Foreign Application Priority Data
Jan. 18, 2007 (FI) 20075030

(51) Int. Cl. C12N 9/10; C12N 5/02; C12N 5/071; B01D 59/44

(52) U.S. Cl. 435/193; 435/377; 435/366; 250/282

(57) ABSTRACT

The invention describes specific sialylated structures present on human stem cells and cell populations derived thereof. The invention is especially directed to methods to control the status of stem cells by changing sialylation and/or fucosylation levels of the cells. The invention is further directed to novel stem cells, the glycosylation of which has been specifically altered. The control methods are preferably mass spectrometric methods.

### FIELD OF INVENTION

The invention describes specific sialylated structures present on human stem cells and cell populations derived thereof. The invention is especially directed to methods to control the status of stem cells by changing sialylation levels of the cells. The invention is further directed to novel stem cells, the glycosylation of which has been specifically altered. The control methods are preferably mass spectrometric methods

### BACKGROUND OF THE INVENTION

### Martin et al.

### 2005, Nat. Med., Publ. Online 30 Jan. 2005, doi 10.1038i

Analysis of sialylated glycans from embryonal stem cells was produced in USA and reported for human embryonal stem cell lines, such cell lines have been reported to be contaminated with N-glycolylneuraminic acid (NeuGc) and amounts of both NeuGc and N-acetylneuraminic acid (NeuAc) have been quantitated. The scientists further discussed cell culture materials containing NeuGc and causing contamination of the cultivated cell lines and for handling the problem by using alternative recombinant protein materials and especially heat inactivated human serum not originally containing NeuGc.

The inventors of the present invention were able to find new sources of potential NeuGc and other sialyl-glycan contaminations. The present invention further describes specific sialyl-glycan structures from early human cells. The inventors were also able to describe specific protein reagents involved in cell production processes, which need to be controlled with regard to glycosylation, especially albumin, gelatine, and antibody reagents.

### Varki, US Patent Document 2005

The patent document describes monosaccharide NeuGc analysis from foods and other materials. There are specific claims for the proportion of NeuGc of the sum of NeuGc and NeuAc, especially in food materials. The document further describes anti-NeuGc antibodies present in patients and production of antibodies involving oxidation of the glycerol tail of NeuGc. The incorporation of NeuGc to a cultured endothelial (cancer) cell line was studied in serum containing culture by adding free NeuGc.

Analysis of CD34+ Hematopoietic Stem Cell Materials with Regard to NeuAcα3Galβ4

NeuAcα3Galβ4 structures have been previously indicated to be present in human cord blood CD34⁺ hematopoietic cells by the use of a specific monoclonal antibody (Magnani, J., et al., U.S. Pat. No. 5,965,457.). The invention claims all CD34+ cells and especially ones from cord blood and bone marrow

The inventors of the present invention were able to analyse, in human stem cell and cord blood cell populations, the presence of both NeuAcα3 and NeuAcα6 structures and even NeuGcα3/6, and larger sialylated structures, including also information about the glycan core structures by which the glycans are linked to the cell. The present invention is in a preferred embodiment directed to analysis of at least two or several sialylated terminal epitopes or at least one whole glycan structure. In a preferred embodiment the sialic acid analysis of cord blood cells is directed to multipotent cell populations, which are not CD34⁺ hematopoietic progenitor cells. Preferably the analysis includes analysis of the core structures of N-linked glycans since Magnani et al. (US pat.) do not describe the core structures by which the glycans are linked to the cells.

### Desialylation and Resialylation of Cells According to the Invention

Changes of sialylation by desialylation and resialylation with specific sialyltransferases has been reported for red cells in order to analyze binding specificities of influenza virus (Paulson, J., et al.).

Partial desialylation and alpha-6-resialylation with CMP-NeuAc-fluorescein of human peripheral blood and bone marrow aspirate-derived CD34+ cells has been reported, the peripheral blood cells having been released by GM-CSF and most of the subjects being under cancer therapy (Schwarz-Albiez, Reihard et al., 2004, Glycoconj. J. 21 451-459). The large variations in results may be due to therapy and GM-CSF. The method used does not reveal real quantitation of sialic acid types due to limited specificity of especially the sialyltransferase used, nor are the possible carrier structures of the sialic acids revealed. The modifications of sialic acid would likely further affect the acceptor specificity of the sialyltransferase used and thus the structures labelled. The present invention is especially directed to α3-sialylation of the specific carrier structures.

Removal of NeuGc from pig xenotransplant tissue and resialylation by NeuAc and sialyltransferase has been also suggested (WO02088351)). That work was not directed to stem cells, nor human stem cells directed methods, nor were the methods used specified, although this is essential for applications in these cells. The xenotransplantation idea is not relevant to present invention due to tissue and species specificity of glycosylation. A patent application (WO2003/105908) describes possible sialidase and sialyltransferase reactions for certain NK/lymphocyte cell lines in a patent application also discussing separately stem cells. The results reveal that the possible reactions vary between cell lines of the same type and are not expected/predictable under the conditions used in the work, possibly partially due to nature of the cells and specificities of enzymes, further the reaction conditions of sialyltransferase without CMP-sialic acid are not described by the invention.

Methods of removal of terminal Gal or GalNAc from human red cells have also been described as well as galactosylation of human platelets in the context of cryopreservation induced changes in human platelets (Zymequest; Science 2004). In context α-galacotosidase/α-galNAcdase reactions to modify erythrocytes, use of an inhibitor for release enzymes after reaction has been indicated, but the chemical nature of the inhibitors were not indicated (patent application Henry et al WO04 Kode/Kiwi NZ). There is no indication of use of inhibitor, especially soluble acceptor mimicking competitive inhibitor in context of cells and sialidase reactions or glycosyltransferase reactions, which are mechanistically different and moreover part of the substrates, especially donor substrates such as such e.g. GDP for fucosyltransferases or CMP for sialyltransferases were realized actually to increase the binding of the enzymes to cells. Furthermore no useful concentration ranges for the substrates has been indicated and the inventors were first to reveal the need of removal of bound glycosyltranferases from enzymes. There is also no indication of chemically conjugated tags nor glycan conjugated tags on enzymes for purification of these in context of cells, though a recombinant erythrocyte modification enzyme was indicated, with a fusion cellulose binding domain was suggested without specific indication of the purification method.

Xia et al (2004) Blood 104 (10) 3091-9 describes changing fucosylation of cord blood cell population without changing sialylation levels and possible usefulness of modification in bone marrow targeting.

None of the reports describe the specific expression of the preferred sialylated N-glycan structures of human stem cells and cord blood cells. It is generally known that glycosylation is cell type specific, and this has been further indicated by the present invention. It cannot be known in advance and based on prior art if the cells contain sialic acid residues removable by specific sialidase enzymes or specific acceptor sites for specific sialyltransferases. Specific sialyltransferases according to the invention, especially recombinant human sialyltransferases controlled with regard to glycosylation, are preferred for the process described in the present invention. The present invention is further directed to the synthesis of the specific sialylated glycan structures according to the present invention, which have not been described in the background publications.

### BRIEF DESCRIPTION OF THE DRAWINGS

FIG. 1. MALDI-TOF mass spectrometric detection of sialylated N-glycans that are indicative of N-glycolylneuraminic acid (Neu5Gc). A. Human embryonic stem cell line, B. mesenchymal stem cell line from bone marrow, C. commercial cell culture medium with serum replacement, D. bovine serum transferrin, E. cell culture medium with fetal bovine serum (FBS), and F. fetuin from fetal bovine serum.

FIG. 2. Fragmentation mass spectrometry of parent ion at m/z 2305.50 corresponding to [M−2H+3Na]⁺ adduct ion of NeuAc₁NeuGc₁Hex₅HexNAc₄. Fragment ions corresponding to loss of NeuAcNa (m/z 1991.97), NeuGcNa (m/z 1975.76), or NeuAcNa+NeuGcNa (m/z 1662.56) are the major fragmentation products. x-axis: mass-to-charge ratio (m/z); y-axis: relative signal intensity in arbitrary units (a.u.); m/z 2205.07: unknown.

FIG. 3. Cord blood mononuclear cell sialylated N-glycan profiles before (light/blue columns) and after (dark/red columns) subsequent broad-range sialidase and α2,3-sialyltransferase reactions. The m/z values refer to Table 3.

FIG. 4. Cord blood mononuclear cell sialylated N-glycan profiles before (light/blue columns) and after (dark/red columns) subsequent α2,3-sialyltransferase and α1,3-fucosyltransferase reactions. The m/z values refer to Table 3.

FIG. 5. α2,3-sialidase analysis of sialylated N-glycans isolated from A. cord blood CD133⁺ cells and B. CD133⁻ cells. The columns represent the relative proportions of a monosialylated glycan signal at m/z 2076 (SA₁) and the corresponding disialylated glycan signal at m/z 2367 (SA₂), as described in the text. In cord blood CD133⁻ cells, the relative proportions of the SA₁ and SA₂ glycans do not change markedly upon α2,3-sialidase treatment (B), whereas in CD133⁺ cells the proportion of α2,3-sialidase resistant SA₂ glycans is significantly smaller than α2,3-sialidase resistant SA₁ glycans (A).

### DESCRIPTION OF THE PRESENT INVENTION

### Methods to Alter (Remove or Reduce or Change) Glycosylation of Cells

### Analysis and Degradative Removal of the Harmful Glycan Structure

The present invention is further directed to degradative removal of specific harmful glycan structures from cell, preferably from desired cell populations according to the invention.

The removal of the glycans or parts thereof occurs preferably by enzymes such as glycosidase enzymes.

In some cases the removal of carbohydrate structure may reveal another harmful structure. In another preferred embodiment the present invention is directed to replacement of the removed structure by less harmful or better tolerated structure more optimal for the desired use.

### Biological Uses of the Cells

It was realized that the novel cells produced by the invention are useful for in vivo targeting experiments and animal trials for testing this. The invention is especially directed to the use of the cells for in vivo imagining trials, in preferred embodiment in animal models such as PET imagining e.g. as described in Min J J et al (2006) Ann Nucl Med 20, (3) 165-70 or Kang W J et al (J Nucl Med 47, 1295-1301.

### Use of Asialo Cells in CFU Culture

The invention revealed that all cell populations were viable (Table 7).

The invention further unexpectedly revealed that the cells with quantitatively reduced sialic acid levels gave higher counts in CFU cell culture assay performed as described in (Kekarainen et al BMC Cell Biol (2006) 7, 30) of modified cord blood mononuclear cells. The invention is especially directed to the use of the desialylated hematopoietic cells for cultivation of blood cell populations (Table 7).

### Novel Methods of Modifying Cells by Removable Enzymes

### Tagged Enzyme

The invention revealed novel effective methods for modifying cells by glycosyl modifying enzymes such as glycosidases and/or glycosyltransferases, when the enzymes are removed from the cell preparations. The invention is especially directed to use of specific tag-structures for the removal of the enzymes from the cells.

### Release of Enzymes by Carbohydrate Enzyme Inhibitors

It is realized that enzymes bind cells by their carbohydrate binding sites such as catalytic sites.

In another embodiment the enzymes are removed by incubating the cells with an inhibitor of the enzymes, preferably an inhibitor binding to the catalytic carbohydrate recognizing site of the enzyme. Preferred inhibitors include monosaccharides and monosaccharide glycosides such as methyl and ethylglycosides and more specific inhibitors, which may be designed based on the catalytic site as transition state inhibitors. Preferred inhibitors for sialidases include competitive low activity inhibitors such as sialic acid, and modified or low cost competing substrates such as NeuAcαOMe, NeuNAcαOEt, sialyl-Lactoses available e.g. from bovine milk or polysialic acid available from bacteria (E. coli, colomnic acid): and higher activity inhibitors such as NeuAc2en (NeuNAc with double bond between 2- and 3-positions) or e.g. higher activity inhibitors specific for limited number of enzymes such as influenza virus neuraminidase inhibitors: Tamiflu (oseltamivir, Roche) or Zanamivir (GSK).

The amount of enzyme inhibitor needed can be estimated by inhibition constants. Competitive monosaccharide glycoside or oligosaccharide inhibitors with low millimoler inhibition (or binding constants) are typically needed in amounts of several fold or order of magnitude larger amounts than the inhibition constant. Typical concentrations for the low affinity inhibitors are of about 1-500 mM, more preferably 1-250 mM, and more preferably 2-100 mM, or 2 to 50 mM, even more preferably from about 2 mM to 20 mM. The lower ranges are preferred to maintain the stability and osmotic condition of the cells stable. Typical concentrations for higher affinity inhibitors are from about 1 pM to about 10 mM, depending about the affinity constants. Preferred concentration for low range micromolar inhibitor are between 10-1000 micromolar. Suitable inhibition concentrations are available from literature.

The invention is directed for removing modification enzyme from modified cells involving a step of incubation of the cells with an inhibitor or substrate of the enzyme. The method preferably further comprises steps of washing cells with a suitable solution such as PBS (phosphate buffered saline or other solution suitable, optionally containing additional amount of inhibitor, and preferably a step of final washing with the solution not comprising the inhibitor.

The inhibitor is in a preferred embodiment a sialidase (neuraminidase) inhibitor, and optionally the method is used together with controlled or tagged sialidase enzyme, preferably in a methods as describe in claims 24-29.

### Combination Methods

In a preferred embodiment the invention is directed to removal of the enzyme by combination of the enzyme tagging with the use of the inhibitors.

### Quantitative Change of Sialylation Levels

The invention revealed that it is possible to change quantitatively the sialylation levels of human cells according to the invention. The signals of monosialylated and disialylated sialic acids of biantennary N-glycan cores were measured by MALDI-TOF mass spectrometry of released non-modified N-glycans. It was observed that the sialylation levels of the N-glycans on cell surface could be increased at least by 15% units and even by about 20% or 25% by sialylation of the cells by sialyltransferase enzyme.

It was also observed that the sialylation levels of the N-glycans on cell surface could be decreased at least by 15% units and even by about 20% or 25% by sialylation of the cells by sialylidase (neuraminidase) enzyme.

The invention is especially directed to the cell populations of quantitatively increased and decreased sialylation levels, especially increased and decreased N-glycan sialylation levels of the preferred human cells.

The invention revealed furthermore that the α3-sialylated cells can be fucosylated to produce increased cells increased in sialylated and fucosylated levels comprising sialyl-Lewis x Neu5Acα3Galβ4(Fucα3)GlcNAc (sLex) and related terminal structures. It is realized that sLex content can be further increased by first resialylating the cells and thus reducing α6-silaylated structures blocking sites for possible sLex epitopes. Such sialyl-Lewis x cells are especially useful for in vivo targeting as the structures produced in low amounts from endogenous Neu5Acα3Galβ4GlcNAc can redirect the cells (Xia et al)

### Desialylation Methods

### Preferred Special Target Cell Type

Effective and specific desialylation methods for the specific cell populations were developed. The invention is specifically directed to desialylation methods for modification of human cord blood cells. The cord blood cells are clearly different of other cell types and no desialylation methods have previously been developed for these cells. Due to cell specific differences any quantitative desialylation methods cannot be generalized from one cell population to another. Thus, any results and data demonstrated by other investigators using other cell types are not applicable to cord blood. The present invention is further directed to desialylation modifications of any human stem cell or cord blood cell subpopulation.

The present invention is specifically directed to methods for desialylation of the preferred structures according to the present invention from the surfaces of preferred cells. The present invention is further directed to preferred methods for the quantitative verification of the desialylation by the preferred analysis methods according to the present invention. The present invention is further directed to linkage specific desialylation and analysis of the linkage specific sialylation on the preferred carbohydrate structures using analytical methods according to the present invention.

The invention is preferably directed to linkage specific α3-desialylation of the preferred structures according to the invention without interfering with the other sialylated structures according to the present invention. The invention is further directed to simultaneous desialylation α3- and α6-sialylated structures according to the present invention.

Furthermore the present invention is directed to desialylation when both NeuAc and NeuGc are quantitatively removed from cell surface, preferably from the preferred structures according to the present invention. The present invention is specifically directed to the removal of NeuGc from preferred cell populations, most preferably cord blood and stem cell populations and from the preferred structures according to the present invention. The invention is further directed to preferred methods according to the present invention for verification of removal of NeuGc, preferably quantitative verification and more preferably verification performed by mass spectrometry.

### Modification of Cell Surfaces of the Preferred Cells by Glycosyltransferases

The inventors revealed that it is possible to produce controlled cell surface glycosylation modifications on the preferred cells according to the invention. The present invention is specifically directed to glycosyltransferase catalysed modifications of N-linked glycans on the surfaces of cells, preferably blood cells, more preferably leukocytes or stem cells or more preferably the preferred cells according to the present invention.

The present invention is directed to cell modifications by sialyltransferases and fucosyltransferases. Two most preferred transfer reactions according to the invention are α3-modification reactions such as α3-sialylation and α3-fucosylations. When combined these reactions can be used to produce important cell adhesion structures which are sialylated and fucosylated N-acetyllactosamines such as sialyl-Lewis x (sLex).

### Sialylation

Possible α6-sialylation has been implied in bone marrow cells and in peripheral blood CD34+ cells released from bone marrow to circulation by growth factor administration, cord blood cells or other stem cell types have not been investigated. Furthermore, the previous study utilized an artificial sialic acid modification method, which may affect the specificity of the sialyltransferase enzyme and, in addition, the actual result of the enzyme reaction is not known as the reaction products were not analysed by the investigators. The reactions are likely to have been very much limited by the specificity of the α6-sialyltransferase used and cannot be considered prior art in respect to the present invention.

The inventors of the present invention further revealed effective modification of the preferred cells according to the present inventions by sialylation, in a preferred embodiment by α3-sialylation.

The prior art data cited above does not indicate the specific modifications according to the present invention to cells from early human blood, preferably cord blood, to cultured mesenchymal stem cells, or to cultured embryonal type cells. The present invention is specifically directed to sialyltransferase reactions towards these cell types. The invention is directed to sialyltransferase catalyzed transfer of a natural sialic acid, preferably NeuAc, NeuGc or Neu-O-Ac, from CMP-sialic acid to target cells.

Sialyltransferase catalyzed reaction according to Formula:

CMP-SA+target cell→SA-target cell+CMP,

Wherein SA is a sialic acid, preferably a natural sialic acid,

preferably NeuAc, NeuGc or Neu-O-Ac and
the reaction is catalysed by a sialyltransferase enzyme preferably by an α3-sialyltransferase
and
the target cell is a cultured stem cell or early human blood cell (cord blood cell).

Preferably the sialic acid is transferred to at least one N-glycan structure on the cell surface, preferably to form a preferred sialylated structure according to the invention

### Fucosyltransferase Reactions

In the prior art fucosyltransferase reactions towards unspecified cell surface structures has been studied

The prior art indicates that human cord blood cell populations may be α3-fucosylated by human fucosyltransferase VI and such modified cell populations may be directed to bone marrow due to interactions with selectins.

### Directing Cells and Selectin Ligands

The present invention describes reactions effectively modifying cord blood cells by fucosyltransferases, especially in order to produce sialylated and fucosylated N-acetyllactosamines on cell surfaces, preferably sLex and related structures. The present invention is further directed to the use of the increased sialylated and/or fucosylated structures on the cell surfaces for targeting the cells, in a preferred embodiment for selectin directed targeting of the cells. The invention is especially directed to the cells for targeting to tissue comprising lectins such as lectins binding to the glycans, preferred target tissues includes hematopoietic tissues, preferably bone marrow (as shown in Xia et al 2004) and targeting to other tissues with constitutive or induced expression of the lectins especially selectins.

### Target Stem Cells

The invention is further directed to α3- and/or α4-fucosylation and/or sialylation modification of stem cells including cultured stem cells, preferably stem cells are embryonal stem cells and mesenchymal stem cells preferably derived either from cord blood or bone marrow or hematopoietic stem cells, preferably derived either from cord blood or bone marrow, preferably CD34+ and/or CD133+ cells.

### Fucosylation of Human Peripheral Blood Mononuclear Cell Populations

In a specific embodiment the present invention is directed to α3-fucosylation of the total mononuclear cell populations from human peripheral blood. Preferably the modification is directed to at least to one protein linked glycan, more preferably to an N-linked glycan. The prior art reactions reported about cord blood did not describe reactions in such cell populations and the effect of possible reaction cannot be known. The invention is further directed to combined increased α3-sialylation and fucosylation, preferably α3-sialylation of human peripheral blood leukocytes. It is realized that the structures on the peripheral blood leukocytes can be used for targeting the peripheral blood leukocytes, preferably to selecting expressing sites such as selectin expressing malignant tissues.

### Methods for Combined Increased α3-Sialylation and α3-Fucosylation

The invention is specifically directed to selection of a cell population from the preferred cell population according to the present invention, when the cell population demonstrate increased amount of α3-sialylation when compared with the baseline cell populations.

The inventors revealed that human cord blood in general is highly α6-sialylated and thus not a good target for α3/4-fucosylation reactions, especially for reactions directed to production of selectin ligand structures.

### Use of Selected Cultured α3-Sialic Acid Expressing Cell Populations

The inventors revealed that specific subpopulations of native cord blood cells express increased amounts of α3-linked sialic acid. Preferred selected cell populations from cord blood for α3/4-fucosylation include CD133+ cells.

Furthermore it was found that cultured cells according to the invention have a high tendency to express α3-sialic acid instead to α6-linked sialic acids. The present invention is preferably directed to cultured mesenchymal stem cell lines, more preferably mesenchymal stem cells from bone marrow or from cord blood expressing increased amounts of α3-linked sialic acid

### Fucosylation of α3-Sialylated Cells

The present invention is preferably directed to fucosylation after α3-sialylation of cells, preferably the preferred cells according to the invention. The invention describes for the first time combined reaction by two glycosyltransferases for the production of specific terminal epitopes comprising two different monosaccharide types on cell surfaces.

### Fucosylation of Desialylated and α3-Sialylated Cells

The present invention is preferably directed to fucosylation after desialylation and α3-sialylation of cells, preferably the preferred cells according to the invention. The invention describes for the first time combined reaction by two glycosyltransferases and a glycosidase for the production of specific terminal epitopes comprised of two different monosaccharide types on cell surfaces.

### Sialylation Methods

### Preferred Special Target Cell Type

### Early Human Blood

Effective specific sialylation methods for the specific cell populations were developed. The invention is specifically directed to sialylation methods for modification of human cord blood cells and subpopulations thereof and multipotent stem cell lines. The cord blood cells are clearly different from other cell types and no sialylation methods have been developed for the cell population. Due to cell specific differences any quantitative sialylation methods cannot be generalized from one cell population to another. The present invention is further directed to sialylation modifications of any human cord blood cell subpopulation.

### Embryonal-Type Cells and Mesenchymal Stem Cells

The methods of present invention are further directed to the methods according to the invention for altering human embryonal-type and mesenchymal stem cells. In a preferred embodiment the modification technologies is directed to cultured cells according to the invention.

### Production of Preferred Sialylated Structures

Present invention is specifically directed to methods for sialylation to produce preferred structures according to the present invention from the surfaces of preferred cells. The present invention is specifically directed to production preferred NeuGc- and NeuAc-structures. The invention is directed to production of potentially in vivo harmful structures on cells surfaces, e.g. for control materials with regard to cell labelling. The invention is further directed to production of specific preferred terminal structure types, preferably α3- and α6-sialylated structures, and specifically NeuAc- and NeuGc-structures for studies of biological activities of the cells.

The present invention is further directed to preferred methods for the quantitative verification of the sialylation by the preferred analysis methods according to the present invention. The present invention is further directed to linkage specific sialylation and analysis of the linkage specific sialylation on the preferred carbohydrate structures using analytical methods according to the present invention.

The invention is preferably directed to linkage specific α3-sialylation of the preferred structures according to the invention without interfering with the other sialylated structures according to the present invention. The invention is preferably directed to linkage specific α6-sialylation of the preferred structures according to the invention without interfering with the other sialylated structures according to the present invention.

The invention is further directed to simultaneous sialylation α3- and α6-sialylated structures according to the present invention. The present invention is further directed for the production of preferred relation of α3- and α6-sialylated structures, preferably in single reaction with two sialyl-transferases.

Furthermore the present invention is directed to sialylation when either NeuAc or NeuGc are quantitatively synthesized to the cell surface, preferably on the preferred structures according to the present invention. Furthermore the invention is directed to sialylation when both NeuAc and NeuGc are, preferably quantitatively, transferred to acceptor sites on the cell surface.

The present invention is specifically directed to the removal of NeuGc from preferred cell populations, most preferably cord blood cell populations and from the preferred structures according to the present invention, and resialylation with NeuAc.

The invention is further directed to preferred methods according to the present invention for verification of removal of NeuGc, and resialylation with NeuAc, preferably quantitative verification and more preferably verification performed by mass spectrometry with regard to the preferred structures.

### Controlled Cell Modification

The present invention is further directed to cell modification according to the invention, preferably desialylation or sialylation of the cells according to the invention, when the sialidase reagent is a controlled reagent with regard of presence of carbohydrate material.

Purification of Cells with Regard to Modification Enzyme

The preferred processes according to the invention comprise of the step of removal of the enzymes from the cell preparations, preferably the sialyl modification enzymes according to the invention. Most preferably the enzymes are removed from a cell population aimed for therapeutic use. The enzyme proteins are usually antigenic, especially when these are from non-mammalian origin. If the material is not of human origin its glycosylation likely increases the antigenicity of the material. This is particularity the case when the glycosylation has major differences with human glycosylation, preferred examples of largely different glycosylations includes: procaryotic glycosylation, plant type glycosylation, yeast or fungal glycosylation, mammalian/animal glycosylation with Galα3Galβ4GlcNAc-structures, animal glycosylations with NeuGc structures. The glycosylation of a recombinant enzyme depends on the glycosylation in the production cell line, these produce partially non-physiological glycan structures. The enzymes are preferably removed from any cell populations aimed for culture or storage or therapeutic use. The presence of enzymes which have affinity with regard to cell surface may otherwise alter the cells as detectable by carbohydrate binding reagents or mass spectrometric or other analysis according to the invention and cause adverse immunological responses.

Under separate embodiment the cell population is cultured or stored in the presence of the modification enzyme to maintain the change in the cell surface structure, when the cell surface structures are recovering from storage especially at temperatures closer physiological or culture temperatures of the cells. Preferably the cells are then purified from trace amounts of the modification enzyme before use.

The invention is furthermore directed to methods of removal of the modification reagents from cell preparations, preferably the modification reagents are desialylation or resialylation reagents. It is realized that soluble enzymes can be washed from the modified cell populations. Preferably the cell material to be washed is immobilized on a matrix or centrifuged to remove the enzyme, more preferably immobilized on a magnetic bead matrix.

However, extraneous washing causes at least partial destruction of cells and their decreased viability. Furthermore, the enzymes have affinity with regard to the cell surface. Therefore the invention is specifically directed to methods for affinity removal of the enzymes. The preferred method includes a step of contacting the modified cells with an affinity matrix binding the enzyme after modification of the cells.

Under specific embodiment the invention is directed to methods of tagging the enzyme to be removed from the cell population. The tagging step is performed before contacting the enzyme with the cells. The tagging group is designed to bind preferably covalently to the enzyme surface, without reduction or without major reduction of the enzyme activity. The invention is further directed to the removal of the tagged enzyme by binding the tag to a matrix, which can be separated from the cells. Preferably the matrix comprises at least one matrix material selected from the group: polymers, beads, magnetic beads, or solid phase surface.

### Tagging of Enzyme for Modification Glycan Controlled Enzymes

The invention is furthermore directed to methods of removal of the modification reagents from cells to be depleted of sialic acid and/or resialylated

The preferred modification reagents are desialylation or resialylation reagents. the reagents are tagged to be able to bind the reagents to solid phases comprising specific binder recognizing the tag, the tag binder combination e.g. on microbeads can be removed.

Preferred tags includes
1) antigens such as peptide FLAG or HA-hemagglutinin peptide tag, or
2) chemical tags such as His-tag or fluoroalkane or
   3) biotin, and
   and Invent ion is directed to known specific binder for these such as specific antibodies for peptides, his-tag binding column for His-TAG, fluoroalkane for hydrogen bond binding of fluoroalkane and avidin or strepavidin for biotin are used.

Preferred modification enzymes and enzymes to be tagged includes sialidase (neuraminidases) such as α3-, α6- and multi specific sialidases and α3-, α6-sialyltransferases for example from mammalian or bacterial origin and specific for type I and/or type II N-acetyllactosamines, preferably type two N-acetyllactosamines and N-glycans especially biantennary and triantennary N-glycans known in the art. The invention is specifically directed to preferred tagged enzymes as substances.

Under specific embodiment the invention is directed to methods of tagging the enzyme to be removed from the cells.

Preferably a sialidase enzyme or sialyltransferase is linked to tag-molecule, the tagged enzyme is reacted with the cells to be remodelled and the enzyme is removed after the reaction by immobilizing the enzyme by binding to a molecule specifically binding to the tag and the modified cell(s) are removed from the immobilized enzyme by filtering the cells with matrix of a molecule specifically binding to the tag, preferred matrixes includes column used for cell purification or magnetic beads used for purification of components from cell mixtures (see protocols or catalogs of Dynal and Miltenyi companies).

The tagging step is preferably performed before contacting the enzyme with the cells. The tagging group is designed to bind preferably covalently to the enzyme surface, without reduction or without major reduction of the enzyme activity. Preferred covalent linkage occurs to amine groups, thiol group or oxidized glycan groups as known from catalogue of Pierce.

The invention is further directed to the removal of the tagged enzyme by binding the tag to a matrix, which can be separated from the cells to be modified. Cells proteins are preferably separated from tag-binder immobilized reagents in aqueous media as known in the art of using the tags. Preferably the matrix comprises at least one matrix material selected from the group: polymers, beads, magnetic beads, or solid phase surface.

### Enzymes Acceptable for Humans for Modification of Reagents or Cells

Under specific embodiment the invention is directed to the use for modification of the cells according to the invention, or in a separate embodiment reagents for processes according to the invention, of a human acceptable enzyme, preferably sialidase or sialyltransferase, which is acceptable at least in certain amounts to human beings without causing harmful allergic or immune reactions. It is realized that the human acceptable enzymes may not be needed to be removed from reaction mixtures or less washing steps are needed for desirable level of the removal. The human acceptable enzyme is in preferred embodiment a human glycosyltransferase or glycosidase. The present invention is separately directed to human acceptable enzyme which is a sialyltransferase. The present invention is separately directed to human acceptable enzyme which is a sialidase, the invention is more preferably directed to human sialidase which can remove specific type of sialic acid from cells.

In a preferred embodiment the human acceptable enzyme is purified from human material, preferably from human serum, urine or milk. In another preferred embodiment the enzyme is recombinant enzyme corresponding to natural human enzyme. More preferably the enzyme corresponds to human natural enzyme corresponds to natural cell surface or a secreted from of the enzyme, more preferably serum or urine or human milk form of the enzyme. Even more preferably the present invention is directed to human acceptable enzyme which corresponds to a secreted form of a human sialyltransferase or sialidase, more preferably secreted serum/blood form of the human enzyme. In a preferred embodiment the human acceptable enzyme, more preferably recombinant human acceptable enzyme, is a controlled reagent with regard to potential harmful glycan structures, preferably NeuGc-structures according to the invention. The recombinant proteins may contain harmful glycosylation structures and inventors revealed that these kinds of structures are also present on recombinant glycosyltransferases, even on secreted (truncated) recombinant glycosyltransferases.

### Quantitative and Qualitative Mass Spectrometric Analysis of Modified Cells and or Reagents

The present invention is further directed to the quantitative and qualitative mass spectrometric analysis of modified cells and/or reagents according to the invention.

The invention is directed to production of qualitative glycome analysis of the cell and/or the reagents including determining the monosaccharide composition obtained for the materials.

The present invention is further directed to quantitative mass spectrometric analysis of the materials according to the invention involving determining the intensities of all or part of the mass spectrometric signals verified to be (reasonably) quantitative with regard to the amount of molecules corresponding to the signals, preferably MALDI-TOF mass spectrometric signals.

The invention is further directed to methods, especially research an development methods, such as product development methods, according to the invention for production of reagents or cells as described by the invention involving step of quantitative and/or qualitative glycome analysis, more preferably both quantitative and qualitative analysis.

### Methods for Labelling Cells According to the Invention

The present invention is further directed to methods involving binding to the preferred structures on early human cells. The method is based on the use of a specific binding molecule, referred as “binder”, which binds a marker structure on surface of a cell population. In a preferred embodiment the present invention is directed to use of a protein binding molecule, more preferably an antibody and most preferably a monoclonal antibody.

Preferred antibodies includes antibodies recognizes a structure NeuGcα3Galβ4Glc(NAc)_{0 or 1} and/or GalNAcβ4[NeuGcα3]Galβ4Glc(NAc)_{0 or 1}, wherein [ ] indicates branch in the structure and ( )_{0 or 1} a structure being either present or absent. In a preferred embodiment the invention is directed to the antibody, which is a monoclonal antibody or human monoclonal antibody.

The present invention is further directed to glycan binding molecules, which recognize glycan marker structures on a cell surface. In a preferred embodiment the binding molecule is a protein, more preferably an enzyme, a lectin or a glycan binding antibody.

Preferred lectins includes the lectin is specific for SAα3Gal-structures, preferably being Maackia amurensis lectin or the lectin is specific for SAα6Gal-structures, preferably being Sambucus nigra agglutinin.

The preferred lectins and binding proteins such as antibodies further includes reagents specifically binding to non-human sialic acid (NeuGc and O-acetylated sialic acids), preferably when expressed on N-glycans as described by the invention. In a specifically preferred reagents includes reagents such as proteins (preferably antibodies, lectins, enzymes) binding and recognizing specifically and/or selectively (allowing separation from contaminant present in the cell culture or other cell environment) non-human sialic acid (NeuGc and O-acetylated sialic acids), more preferably O-acetylated sialic acids.

The invention is further directed to search of binding reagents for NeuGc, when the stem cell material according to the invention is not embryonal stem cells and it is preferably differentiated cell derived from embryonal and/or embryonal type stem cells or adult stem cells such as early human cells, or early human blood cells or more preferably blood related stem cells, or cord blood cells or mesenchymal stem cells.

The invention is further directed to development methods, especially research and development methods, such as product development methods, according to the invention for production of reagents or cells as described by the invention involving
i) step of testing a binding reagent against a glycan structure according to the invention and
ii) a step of testing the binding reagent for binding to the cell material, including non-modified and modified cell material, according to the invention.

The invention is further directed to testing methods for selecting optimal and/or most effective and/or -optimal for a specific cell material-binding reagents from reagents known to have suitable specificity allowing recognition of preferred structures according to the invention. Most preferred reagents to be tested includes antibodies, preferably monoclonal antibodies and lectin recognizing same or similar terminal monosaccharide residues structures, preferably involving potential binding to preferred oligosaccharide (involving a preferred disaccharide or trisaccharide epitope) or glycan sequences according to the invention. The invention is specifically directed to known reagents recognizing non-human sialic acid according to the invention.

In a preferred embodiment the invention is directed to testing of human autoimmunity and/or cancer associated antibodies and or lectins such as Cancer antennarius (EY Laboratories, CA, USA) lectin known to recognize O-acetylated sialic acids. The invention is directed especially to the use of Cancer antennarius (EY-laboratories, CA, USA) lectin to recognize cells according to the invention, when there is a risk of contamination by O-acetylated sialic acids and in context of mesenchymal stem cells. The lectin tested for binding against mesenchymal stem cells derived from human bone marrow by lectin labelling methods described in examples, the results revealed that the lectin can recognize sialyl structures on carrier glycans present on the human mesenchymal stem cells.

The invention is especially directed to quantitative determination of o-acetylated sialic levels from human cell populations according to the invention, by mass spectrometric profiling as described here and previous PCT application of the inventor about sialic acid contaminations filed in July 2006, or by labelling by specific binder structures which is included as reference of US proceedings; or by labelling by specific binder structures. The quantitative determination is preferably determination of percent amount of the Neu-OAc molecules of the total sialic acids.

### Preferred Glycan Controlled Reagents and Processes for Preparation Thereof

Preferred reagents to be controlled include preferably all reagents derived from or produced in connection with biological material; preferably these include all glycoprotein, protein mixture, serum, and albumin preparations present in the process. The inventors found out that albumins known to be non-glycosylated proteins may still contain sufficient glycoproteins for contamination of cell material.

In a preferred embodiment the present invention is directed to the control of animal albumins, preferably bovine serum albumin, and human serum albumin preparations for potential contamination by glycan structures.

Other preferred controlled reagents includes controlled transferrin and other serum proteins, even more preferably controlled serum proteins are controlled antibody preparations, preferably Fc blocking antibody preparations.

In yet another embodiment the invention is directed to the production of glycan depleted and/or remodelled protein mixtures preferably glycan remodelled human or animal serum, more preferably a serum from an animal used for production of serum products, preferably cell culture serum or antibodies. Preferred serums to be modified includes serum of cow, horse, sheep, goat, rabbit, rat or mouse, more preferably serum of cow, horse, or sheep, even more preferably fetal bovine serum.

In a preferred embodiment the glycosylation of the serum is altered by a method based on animals with genetically altered glycan production preferably obtained by a) genetic manipulation of the animal or b) breeding a natural or selecting a natural variant of the production animal to used for serum production, preferably the genetic alteration is directed to tissues producing serum proteins.

Controlled Enzyme Preparations for Products Aimed for Use with Transplantable Cells

The present invention is directed under specific embodiment to methods for removal of non-desired carbohydrate structures from living cells. The enzyme proteins are usually antigenic, especially when these are from non-mammalian origin, such as bacteria and/or plants. If the material is not of human origin its glycosylation likely increases the antigenicity of the material. This is particularly the case when the glycosylation has large differences with human glycosylation, preferred examples of largely different glycosylations include: procaryotic glycosylation, plant type glycosylation, yeast or fungal glycosylation, mammalian/animal glycosylation with Galα3Galβ4GlcNAc-structures, animal glycosylation with NeuGc structures. The glycosylation of a recombinant enzyme depends on the glycosylation of the production cell line, these produce partially non-physiological glycan structures in most cases.

### Preferred Classes of Controlled Reagents

### 1. Glycan Depleted Biological Materials, Preferably Glycoprotein Materials

Present invention is specifically directed to use biological materials, preferably glycoprotein material, from which harmful structure is removed or reduced in amount. Glycoproteins are major source of bioactive glycans, in some material presence of glycolipids may be also possible and could be handled similarly. In case the lipid part of glycolipid binds it to the material, released glycan or part of it is water soluble and can be separated. The invention is further directed to glycan depletion methods. In a preferred embodiment the invention is directed to methods including steps of releasing glycan structure and removing released glycan structure.

Preferred methods for removal of the released glycan structure include filtration methods. The filtration methods are based on size difference of the released glycan structure and the glycan depleted protein. A preferred method for removal of the released glycans includes precipitation methods, in a preferred embodiment the invention is directed to precipitation of the protein under conditions where the released glycan structure is soluble.

The glycan depletion may be combined with a step of inactivation of potential harmful proteins such as lectins or antibodies possibly involved in the process. Some reagents such serum in certain cell culture processes may be heat inactivated. The inactivation may be partial. The partial inactivation is in a preferred embodiment performed by releasing glycans inhibiting the harmful binding proteins to the reagent and further to cell involving process. In a preferred embodiment the depleted glycan and the binding protein inhibiting glycan is the same structure. Preferably the released glycans are used when these can not be incorporated to cells to cause further problems in the cell related process. The method of released glycans is not preferred for NeuGc under conditions where it can be incorporated to cells.

Terminally depleted glycans. In a preferred embodiment one or several terminal structures are depleted from a biological material, preferably glycoprotein material. The preferred methods to deplete terminal structures include enzymatic and chemical methods. Preferred enzymatic method is hydrolysis by a glycosidase enzyme or by a trans-glycosylating enzyme capable of removing the terminal structure. Terminal depletion may further include release of several terminal monosaccharide units for example by glycosidase enzymes. Preferred chemical hydrolysis is an acid hydrolysis, preferably a mild acid hydrolysis under conditions not destroying protein structure or from which the protein structure can be restored or renatured. The structure to be depleted is in a preferred embodiment a sialic acid. The sialic acid is preferably released by a sialidase enzyme or by mild acid hydrolysis.

Internally depleted glycans. The present invention is further directed to internal depletion of glycan material by release of glycans from subterminal linkages by chemical and/or enzymatic methods. Methods to release glycans chemically include base hydrolysis methods such as beta elimination for release of O-linked glycans, hydrazinolysis methods to release O-glycans and N-glycans, oxidative methods such as Smith degradation and ozonolysis (preferred for glycolipids). Preferred enzymatic methods includes use of endo-glycosidases such as endoglycosylceramidase for glycolipids, N-glycosidases for N-glycans, and O-glycosidases for O-glycans.

2. Glycosylated Reagents from Non-Animal Sources

In a preferred embodiment the present invention is directed to the use of reagents from non-animal sources devoid of potentially harmful reagents. Preferred non-animal glycosylated proteins are proteins from yeasts and fungi and from plants. It is notable that even these materials contain glycans, which may have harmful allergenic activities or which may cause problems in analysis of human type glycans. Preferably the invention is further directed to control of the glycosylated reagents from non-animal structures, too. Preferred plant derived proteins include recombinant albumins produced by plant cell culture, more preferably non-glycosylated human serum albumins and bovine serum albumins and recombinant gelatin materials such as collagens produced by plant cell systems. The present invention is specifically directed to the processes according to present invention, when a material containing glycans or harmful glycans according to the present invention is replaced by a reagent, preferably a controlled reagent from non-animal sources.

3. Non Glycosylated Reagents from Procaryotes

Many bacterial recombinant proteins are known for lacking expression of glycans. Present invention is directed to control of glycosylation of bacterial protein, as this happens on certain proteins. The present invention is specifically directed to the processes, when a material containing glycans or harmful glycans according to the present invention is replaced by a reagent, preferably a controlled reagent from procaryotes.

Under specific embodiment the present invention is directed to use of glycan controlled forms of glycosidase enzymes for modification of transplantable cells according to the invention and removal of the enzymes from reactions as described by the present invention

The present invention is also specifically directed to the glycan controlled enzyme preparations, especially when produced in a mammalian cell line/cultivation process and controlled with regard to Galα3Galβ4GlcNAc-structures, animal glycosylations with NeuGc structures. The preferred enzymes are of human origin, more preferably recombinant enzymes. Most preferably a human serum form of the enzyme is selected and the glycosylation is controlled to be a non-antigenic human-type glycosylation, preferably similar to the glycosylation human natural soluble enzyme.

Preferred Viability Levels of Cells after the Enzyme Reactions

The invention is preferably directed to the novel cell populations produced according to the invention, wherein the cell viability is over 90% in comparison to non-treated control, more preferably over 93%, more preferably over 94%, more preferably over 95%, more preferably over 96%, more preferably over 97%, and most preferably over 98%. The viability data is shown in Table 7 and in example 8 shows also values close to or even over the control. The handling of the cell preparation before the experiment control cells was not optimal.

### Conjugated Enzymes

The present invention is directed to the use of the specific enzyme for or in context of modification of the stem cells wherein the enzyme is covalently conjugated to a tag. The conjugation according to the invention may be performed non-specifically e.g. by biotinylation one or several of multiple amines on cells surface or specifically.

### Specific Conjugation

The specific conjugation aims for conjugation from protein regions, which does not disturb the binding of the binding site of the enzyme to its ligand glycan and/or donor nucleotide binding site of a glycosyltransferase to be modified on the cell surface glycans of stem cells according to the invention.

Preferred specific conjugation methods includes chemical conjugation from specific amino acid residues from the surface of the enzyme protein/peptide. In a preferred method specific amino acid residue such as cysteine is cloned to the site of conjugation and the conjugation is performed from the cysteine. In another preferred method N-terminal cysteine is oxidized by periodic acid and conjugated to aldehyde reactive reagents such as amino-oxy-methyl hydroxylamine or hydrazine structures, further preferred chemistries includes “Click” chemistry marketed by Invitrogen and amino acid specific coupling reagents marketed by Pierce and Molecular probes.

A preferred specific conjugation occurs from protein linked carbohydrate such as O- or N-glycan of the enzyme, preferably when the glycan is not close to the binding site of enzyme substrates or longer spacer is used.

### Glycan Conjugated Enzyme Protein

Preferred glycan conjugation occurs through a reactive chemoselective ligation group R1 of the glycans, wherein the chemical group can be specifically conjugated to second chemoselective ligation group R2 without major or binding destructive changes to the protein part of the enzyme. Chemoselective ligation groups reacting with aldehydes and/or ketones include as amino-oxy-methyl hydroxylamine or hydrazine structures. A preferred R1-group is a carbonyl such as an aldehyde or a ketone chemically synthesized on the surface of the protein. Other preferred chemoselective groups includes maleimide and thiol; and “Click”-reagents (marketed by Invitrogen) including azide and reactive group to it.

Preferred synthesis steps includes
a) chemical oxidation by carbohydrate selectively oxidizing chemical, preferably by periodic acid or
b) enzymatic oxidation by non-reducing end terminal monosaccharide oxidizing enzyme such as galactose oxidase or by transferring a modified aldehyde or ketone group comprising monosaccharide residue (such as Gal Comprising CH₃COCH₂— instead of OH on position 2) to the terminal monosaccharide of the glycan.

Use of oxidative enzymes or periodic acid are known in the art has been described in patent application directed conjugating HES-polysaccharide to recombinant protein by Kabi-Frensenius (WO2005EP02637, WO2004EP08821, WO2004EP08820, WO2003EP08829, WO2003EP08858, WO2005092391, WO2005014024 included fully as reference) and a German research institute.

Preferred methods for the transferring the terminal monosaccharide reside includes use of mutant galactosyltransferase as described in patent application by part of the inventors US2005014718 (included fully as reference) or by Qasba and Ramakrishman and colleagues US2007258986 (included fully as reference) or by using method described in glycopegylation patenting of Neose (US2004132640, included fully as reference).

### Conjugates Including High Specificity Chemical Tag

In a preferred embodiment the enzyme is, specifically or non-specifically conjugated to a tag, referred as T, specifically recognizable by a ligand L, examples of tag includes such as biotin biding ligand (strept)avidin or a fluorocarbonyl binding to another fluorocarbonyl or peptide/antigen and specific antibody for the peptide/antigen

### Tag-Conjugate Structures

The preferred conjugate structures are according to the

B-(G-)ₘR1-R2-(S1-)ₙT-L-(S2)ₛ-SOL, Formula CONJ

Wherein B is the enzyme, SOL is solid phase or affinity matrix or polymer or other matrix useful for removal of the enzyme, G is glycan (when the enzyme is glycan conjugated), R1 and R2 are chemoselective ligation groups, T is tag, preferably biotin, L is specifically binding ligand for the tag; S1 and S2 are optional spacer groups, preferably C₁-C₁₀ alkyls, m, n, p, r and s are integers being either 0 or 1, independently.

Methods to chemically attach spacer structures ligation groups or ligand such as (strept)avidin to solid phases is known in the art.

### Complex Structure

When the enzyme is removed by using the tag following complex structure is preferably formed

B-(G-)ₘR1-R2-(S1-)ₙT-L-(S2)ₛ-SOL,

Wherein B is the enzyme, SOL is solid phase or affinity matrix or polymer or other matrix useful for removal of the enzyme, G is glycan (when the enzyme is glycan conjugated), R1 and R2 are chemoselective ligation groups, T is tag, preferably biotin, L is specifically binding ligand for the tag; S1 and S2 are optional spacer groups, preferably C₁-C₁₀ alkyls, m, n, and s are integers being either 0 or 1, independently and linkage between T-L can be non-covalent high affinity binding.

Methods to chemically attach spacer structures or ligand such as (strept)avidin to solid phases is known in the art.

### Use of the Tag Conjugates

A preferred method of the tag conjugate involves following steps:

1) incubating the tagged enzyme with cells
2) optional addition of enzyme inhibitor for the release of the enzyme from the cells
3) Contacting the releases tagged enzyme with a matrix comprising the specific ligand for the tag
4) Isolating the enzyme-matrix complex from the cells

The matrix comprising the ligand may be solid phase or affinity matrix or polymer or other matrix useful for removal of the enzyme. The matrix may be used in form of magnetic particles, column, surface of tubing or vessel, soluble or insoluble preferably water miscible polymer.

In yet another preferred embodiment the tagged enzyme is used together with non-tagged enzyme in order to establish the level of non-tagged enzyme with same or very similar cell binding properties in a cell preparation, preferably aimed for therapeutic use, and removal of the tagged enzyme.

### Modification of Mesenchymal Stem Cells

In a preferred embodiment the invention is directed to modification of mesenchymal stem cells, preferably selected from the group blood tissue or cell derived mesenchymal stem cells such as cord blood mesenchymal stem cells or bone marrow mesenchymal stem cells. Preferably mesenchymal stem cells are modified to increase sialylation and/or fucosylation as a combination method, preferably by preferred transferases for sialyl-LacNAc synthesis and fucosylation such as STGalIII and Fuc-TVI.

Preferred fucosyltransferase conditions includes about 4 mU (especially for Fuc-TVI, Calbiochem enzyme and units, fresh enzyme) per 3 million cells, or from 0.5 to 5 mU per million cells, more preferably 0.75-3 mU, and most preferably 1-2 mU per million cells. The preferred range depend on the status of the enzyme (decays during storage) and status and type of the cells. The preferred reaction temperature is about 37 degrees of Celsius, preferably between 33-40 Degrees of Celsius and more preferably 35-39 degrees of Celsius. The preferred reaction times varies from 0.5 to 6 hours preferably between 1-6 hours, more preferably between 2-6 hours, even more preferably between 3-5.5 hours and in a preferred embodiment about 4 hours (3.5-4.5 hours). It is realized that increasing the enzyme amount reduces reaction time needed.

Preferred sialyltransferase conditions includes about 50 mU (especially for α2,3-(N)-Sialyltransferase (Calbiochem), Calbiochem enzyme and units, fresh enzyme) per 1 million cells, or from 5 to 200 mU per million cells, more preferably 10-150 mu, and most preferably 25-75 mU per million cells. The preferred range depend on the status of the enzyme (decays during storage) and status and type of the cells. The preferred reaction times varies from 0.5 to 6 hours preferably between 1-6 hours, more preferably between 2-6 hours, even more preferably between 3-5.5 hours and in a preferred embodiment about 4 hours (3.5-4.5 hours). The preferred reaction temperature is about 37 degrees of Celsius, preferably between 33-40 Degrees of Celsius and more preferably 35-39 degrees of Celsius. It is realized that increasing the enzyme amount reduces reaction time needed.

The invention is especially directed to modification of stem cells especially mesenchymal stem cells wherein the cells have unusually low sialylation levels. The cells with low sialylation comprise more than 30% of N-glycans in non-sialylated form. In a preferred embodiment the mesenchymal stem cell with low sialylation is a bone marrow derived mesenchymal stem cell.

### Preferred Glycosylation Levels for Modification of Cells

The present invention revealed that it is possible glycosylate cells preferably to silylate cells to over 50% level of available free sialylation sites on N-glycans (when calculated based on the disappearance of the sialylation sites). In a preferred embodiment the invention is directed to sialylation by single sialyltransferase to level over 60% more preferably over 70% even more preferably over 75%, even more preferably over 80% or at least 83% and most preferably over 85%. The invention is further directed to a novel mesenchymal stem cell population comprising increased sialylation of over 60% more preferably over 70% even more preferably over 75%, even more preferably over 80% or at least 83% and most preferably over 85%. The cell population is preferably derived from human cord blood or bone marrow.

In a preferred embodiment the optimized sialylation is performed on cord blood mesenchymal stem cells.

### Preferred Sialyltransferases

Preferred sialyltransferases includes mammalian, more preferably human α3-, and α6-sialyltransferases, preferably in soluble form. In a preferred embodiment the transferase sialylates N-acetyllactose amines such as ST3GalIII and ST3GalIV or ST6GalI or O-glycans core I such as ST3GalI or ST3GalII and ST3GalIV. It is realized that most effective sialylation is obtained with combination of at least two sialyltransferases such as core I sialylating and N-acetyllactosamine sialylating, e.g. ST3GalIII and ST3GalIV or ST3GalI/II and ST3GalIV.

### Preferred Fucosyltransferases

Preferred fucosyltransferases includes mammalian, more preferably human α3-, and α6-fucosyltransferases, preferably in soluble form. In a preferred embodiment the transferase reacts with N-acetyllactosamines such as FTIII, FTIV, FTV, FTVI, FTVII and FTIX more preferably sialylα3-N-acetyllactosamines, preferably FTIII, FTIV, FTV, FTVI, FTVII, more preferably FTIII, FTV, FTVI, and FTVII, even more preferably FTVI, and FTVII, and most preferably FTVI. It is realized that most effective fucosylation is obtained with combination of at least two fucosylatrasferases.

In a preferred embodiment the galactosylation reaction is performed in the presence Mg2+ ions as described in US2005014718 (included fully as reference), preferably by mammalian GalT, more preferably natural human GalT, or using exogenous transferase such as Mg2+ selective β4-Galactosyltransferase of Qasba and Ramakrishnan.

It is realized that it is useful to remove exogenous GalT and/or sialyltransferase by using specific Tags according to the invention and/or by using enzyme inhibitors according to the invention.

### Controlled Reagents for the Modification

In another preferred embodiment it is useful to use glycan controlled sialyltransferase or galactosyltransferase. The invention is directed to analysis of glycans of non-human expressed glycosyltransferases. When the transferases comprise non-human glycosylation, the non-human structures are preferably removed by specific glycosidases or modified by chemically e.g. by periodate oxidation and reduction

The invention is especially directed to use of controlled enzyme substances, preferably galactosyltransferase or sialyltransferase for reaction according to the invention comprising glycan according to Formula

Manα6(Manα3)Manβ4GlcNAcβ4(Fucα6)ₙGlcNAc-N-E

wherein E is enzyme protein, N is glycosidic linkage nitrogen in N-glycosylation site (Asn-X-Ser/Thr) a

And the non-reducing end mannoses may be further modified by (NeuNAcα3/6)ₘGalβ4GlcNAcβ2, wherein m and n are 0 or 1

If the enzyme would comprise NeuNGc instead of NeuNAc, this is preferably removed and changed to NeuNAc.

In another preferred embodiment the enzymes are non-glycosylated preferably from bacterial production e.g. as described by Qasba US2007258986 (included fully as reference) or N-(Asn-X-Ser/Thr/Cys) and possible O-glycosylation sites of the enzymes are mutated for expression in eukaryotic system.

The structure is especially beneficial because Manβ4-residue is devoid of Xylβ2-modification present in plant cells and reducing end GlcNAc is devoid of Fucα3-structure present in insect or plant cell derived material (e.g. when the enzyme would be produced by insect or plant cell culture).

Glycosyltransferase Inhibitors for Release of Glycosyltransferase from Cells

The present invention is especially directed to use of analogs or derivatives of acceptor saccharides or donor nucleotides for inhibitors of glycosyltransferases for washing the transferase effectively from cells after the reaction. The preferred acceptor analogs includes carbohydrates oligosaccharides, monosaccharides and conjugates and analogs thereof capable of binding to substrate site and inhibiting the acceptor binding of the enzyme. The preferred concentrations of the carbohydrates includes concentrations tolerable by the cells from 1 mM to 500 mM, more preferably 5 mM to 250 mM and even more preferably 10-100 mM, higher concentrations are preferred for monosaccharides and method involving solid phase bound binders.

Preferred oligosaccharide for sialyltransferase inhibition includes sequences including oligosaccharides and reducing end conjugates includes Galβ4Glc, Galβ4GlcNAc, Galβ3GlcNAc, Galβ3GalNAc depending. GalT inhibitors includes GlcNAc and conjugates and GlcNAcβ2Man, GlcNAcβ6Gal and GlcNAcβ3Gal.

In a preferred embodiment sialyltransferase is released by acceptor disaccharide, more preferably by 5-150 mM acceptor, more preferably by 10-100 mM, even more preferably 10-80 mM, more preferably 10-50 mM for high affinity acceptor and 20-100 mM, more preferably 40-100 mM, most preferably 50-100 mM for low affinity acceptor. It is realized that acceptor affinities varies between enzymes, lactose is considered as medium low affinity acceptor for α2,3-(N)-Sialyltransferase (Calbiochem) or ST3GalIII and high affinity acceptors have typically acceptor Km values about 10 fold lower. Preferably washing removes at least 50% of the cell bound enzyme even more preferably at least, 70%, even more preferably at least 85%, even more preferably at least 90% and most preferably at least 95%.

The preferred reducing end structure in conjugates is

AR, wherein A is anomeric structure preferably beta for Galβ4Glc, Galβ4GlcNAc, Galβ3GlcNAc, and alfa for Galβ3GalNAc and R is organic residue linked glycosidically to the saccharide, and preferably alkyl such as method, ethyl or propyl or ring structure such as a cyclohexyl or aromatic ring structure optionally modified with further functional group. Preferred monosaccharides includes terminal or two or three terminal monosaccharides of the binding epitope such as Gal, GalNAc, GlcNAc, Man, preferably as anomeric conjugates: as FucαR, GalβR, GalNAcβR, GalNAcαR GlcNAcβR, ManαR. For example α3- or α6-sialyltransferase synthesizing sialyl Galβ4GlcNAc is preferably inhibited by Galβ4GlcNAc or lactose. Preferred donor analog includes CMP and derivatives for sialyltransferases and UDP and derivatives for galactosyltransferases, the analogs preferably interfere also with acceptor binding so that the enzyme is released.

### Sialyltransferase Catalyzed Transfer of a Natural Sialic Acid

The invention is directed to sialyltransferase catalyzed transfer of a natural sialic acid, preferably NeuAc, NeuGc or Neu-O-Ac, from CMP-sialic acid to target cells.

The invention provides sialyltransferase catalyzed reaction according to Formula

CMP-SA+target cell→SA-target cell+CMP,

preferably

CMP-SA+Galβ4/3GlcNAc-target cell→SAα3/6Galβ4/3GlcNAc-target cell+CMP,

wherein SA is a sialic acid, preferably a natural sialic acid,
preferably NeuAc, NeuGc or Neu-O-Ac and
the reaction is catalysed by a sialyltransferase enzyme preferably by an α3-sialyltransferase
and
the target cell is a cultured stem cell or stem cell or early human blood cell (cord blood cell).

Preferred fucosyltransferase reactions synthesis of Lewis a and Lewis x and sialylated variant thereof are:

GDP-Fuc+Galβ4/3GlcNAc-target cell→Galβ4/3(Fucα3/4)GlcNAc-target cell+GDP,

and/or

GDP-Fuc+SAα3Galβ4/3GlcNAc-target cell→SAα3Galβ4/3(Fucα3/4)GlcNAc-target cell+GDP,

Both synthesis of sialyl-Lewis x, SAα3Galβ4(Fucα3)GlcNAc, and sialyl-Lewis a, SAα3Galβ3(Fucα4)GlcNAc, are preferred, sLex more preferred, when the cells comprise mainly type 2 lacNAc acceptors common on mesenchymal stem cells.

The reaction is catalysed by a fucosyltransferase enzyme preferably by an α3/4-fucosyltransferase. α4-fucosyltransferases (Fuc-TIII and -TV) are preferred for synthesis of Lewis a. The novel fucosylated cell populations are preferred for functional studies of the structure.

### EXAMPLES

### Example 1

### Detection of N-Glycolylneuraminic Acid Containing Glycan Structures in Stem Cell and Differentiated Cell Samples, Cell Culture Media, and Biological Reagents

### Examples of Cell Material Production

### Cord Blood Cell Populations

Preparation of mononuclear cells. Cord blood was diluted 1:4 with phosphate buffered saline (PBS)-2 mM EDTA and 35 ml of diluted cord blood was carefully layered over 15 ml of Ficoll-Paque® (Amersham Biosciences, Piscataway, USA). Tubes were centrifuged for 40 minutes at 400 g without brake. Mononuclear cell layer at the interphase was collected and washed twice in PBS-2 mM EDTA. Tubes were centrifuged for 10 minutes at 300 g.

Positive selection of CD34+/CD133+ cells. The cord blood mononuclear cell pellet was resuspended in a final volume of 300 μl of PBS-2 mM EDTA-0.5% BSA (Sigma, USA) per 10⁸ total cells. To positively select CD34+ or CD133+ cells, 100 μl of FcR Blocking Reagent and 100 μl CD34 or CD133 Microbeads (Miltenyi Biotec, Bergisch Gladbach, Germany) were added per 10⁸ mononuclear cells. Suspension was incubated for 30 minutes at 6-12° C. Cells were washed with PBS-2 mM EDTA-0.5% BSA and resuspended in 500 μl of PBS-2 mM EDTA-0.5% BSA per 10⁸ cells.

The appropriate MACS affinity column type (Miltenyi Biotec, Bergisch Gladbach, Germany) was chosen according to the number of total cells: MS column for <2×10⁸ cells and LS column for 2×10⁸-2×10⁹ cells. The column was placed in the magnetic field and rinsed with PBS-2 mM EDTA-0.5% BSA. Labeled cell suspension was applied to the column and the cells passing through the column were collected as the negative cell fraction (CD34− or CD133−). The column was then washed four times with PBS-2 mM EDTA-0.5% BSA. The column was removed from the magnetic field and the retained positive cells (CD34+ or CD133+) were eluted with PBS-2 mM EDTA-0.5% BSA using a plunger.

The eluted positive cells were centrifuged for 5 minutes at 300 g and resuspended in 300 μl PBS-2 mM EDTA-0.5% BSA. 25 μl of FcR Blocking Reagent and 25 μl CD34 or CD133 Microbeads were added. Suspension was incubated for 15 minutes at 6-12° C. Cells were washed with PBS-2 mM EDTA-0.5% BSA and resuspended in 500 μl of PBS-2 mM EDTA-0.5% BSA.

A MS column was placed in the magnetic field and rinsed with PBS-2 mM EDTA-0.5% BSA. Labeled cell suspension was applied to the column. The column was washed four times with PBS-2 mM EDTA-0.5% BSA. The column was then removed from the magnetic field and the retained positive cells (CD34+ or CD133+) were eluted with PBS-2 mM EDTA-0.5% BSA using a plunger.

Negative selection of Lin− cells. To deplete lineage committed cells, mononuclear cells (8×10⁷/ml) in PBS-0.5% BSA were labeled with 100 μl/ml cells with StemSep Progenitor Enrichment Cocktail containing antibodies against CD2, CD3, CD14, CD16, CD19, CD24, CD56, CD66b, Glycophorin A (StemCell Technologies, Vancouver, Canada) at room temperature for 15 minutes. Subsequently, 60 μl of colloidal magnetic iron particles were added per 1 ml cell suspension and incubated at room temperature for 15 minutes.

The labeled cell suspension was loaded into MACS LD column (Miltenyi Biotec) and unlabeled cells passing through the column were collected as the negative fraction (Lin−). LD column was washed twice with 1 ml PBS-0.5% BSA and effluents were collected into the same tube with unlabelled cells. The column was then removed from the magnetic field and the retained positive cells (Lin+) were eluted with PBS-0.5% BSA using a plunger.

### Cord Blood Mesenchymal Stem Cell Lines

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400 g/40 min). The mononuclear cell fragment was collected from the gradient and washed twice with PBS.

Umbilical cord blood cell isolation and culture. CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements.

The cells were initially cultured in media consisting of 56% DMEM low glucose (DMEM-LG, Gibco, http:www.invitrogen.com) 40% MCDB-201 (Sigma-Aldrich) 2% fetal calf serum (FCS), 1× penicillin-streptomycin (both form Gibco), 1×ITS liquid media supplement (insulin-transferrin-selenium), 1× linoleic acid-BSA, 5×10⁻⁸ M dexamethasone, 0.1 mM L-ascorbic acid-2-phosphate (all three from Sigma-Aldrich), 10 nM PDGF (R&D systems, http://www.RnDSystems.com) and 10 nM EGF (Sigma-Aldrich). In later passages (after passage 7) the cells were also cultured in the same proliferation medium except the FCS concentration was increased to 10%.

Plates were screened for colonies and when the cells in the colonies were 80-90% confluent the cells were subcultured. At the first passages when the cell number was still low the cells were detached with minimal amount of trypsin/EDTA (0.25%/1 mM, Gibco) at room temperature and trypsin was inhibited with FCS. Cells were flushed with serum free culture medium and suspended in normal culture medium adjusting the serum concentration to 2%. The cells were plated about 2000-3000/cm². In later passages the cells were detached with trypsin/EDTA from defined area at defined time points, counted with hematocytometer and replated at density of 2000-3000 cells/cm².

Isolation and culture of bone marrow derived stem cells. Bone marrow (BM)-derived MSCs were obtained as described by Leskelä et al. (2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

Flow cytometric analysis of mesenchymal stem cell phenotype. Both UBC and BM derived mesenchymal stem cells were phenotyped by flow cytometry (FACSCalibur, Becton Dickinson). Fluorescein isothiocyanate (FITC) or phycoerythrin (PE) conjugated antibodies against CD13, CD14, CD29, CD34, CD44, CD45, CD49e, CD73 and HLA-ABC (all from BD Biosciences, San Jose, Calif., http://www.bdbiosciences.com), CD105 (Abcam Ltd., Cambridge, UK, http://www.abcam.com) and CD133 (Miltenyi Biotec) were used for direct labeling. Appropriate FITC- and PE-conjugated isotypic controls (BD Biosciences) were used. Unconjugated antibodies against CD90 and HLA-DR (both from BD Biosciences) were used for indirect labeling. For indirect labeling FITC-conjugated goat anti-mouse IgG antibody (Sigma-aldrich) was used as a secondary antibody.

The UBC derived cells were negative for the hematopoietic markers CD34, CD45, CD14 and CD133. The cells stained positively for the CD13 (aminopeptidase N), CD29 (β1-integrin), CD44 (hyaluronate receptor), CD73 (SH3), CD90 (Thy1), CD105 (SH2/endoglin) and CD 49e. The cells stained also positively for HLA-ABC but were negative for HLA-DR. BM-derived cells showed to have similar phenotype. They were negative for CD14, CD34, CD45 and HLA-DR and positive for CD13, CD29, CD44, CD90, CD105 and HLA-ABC.

Adipogenic differentiation. To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/ml insulin, 0.1 mM indomethacin, 0.1 μM dexamethasone (Sigma-Aldrich) and penicillin-streptomycin (Gibco) before samples were prepared for glycome analysis. The medium was changed twice a week during differentiation culture.

Osteogenic differentiation. To induce the osteogenic differentiation of the BM-derived MSCs the cells were seeded in their normal proliferation medium at a density of 3×10³/cm² on 24-well plates (Nunc). The next day the medium was changed to osteogenic induction medium which consisted of α-MEM (Gibco) supplemented with 10% FBS (Gibco), 0.1 μM dexamethasone, 10 mM β-glycerophosphate, 0.05 mM L-ascorbic acid-2-phosphate (Sigma-Aldrich) and penicillin-streptomycin (Gibco). BM-derived MSCs were cultured for three weeks changing the medium twice a week before preparing samples for glycome analysis.

Cell harvesting for glycome analysis. 1 ml of cell culture medium was saved for glycome analysis and the rest of the medium removed by aspiration. Cell culture plates were washed with PBS buffer pH 7.2. PBS was aspirated and cells scraped and collected with 5 ml of PBS (repeated two times). At this point small cell fraction (10 μl) was taken for cell-counting and the rest of the sample centrifuged for 5 minutes at 400 g. The supernatant was aspirated and the pellet washed in PBS for an additional 2 times.

The cells were collected with 1.5 ml of PBS, transferred from 50 ml tube into 1.5 ml collection tube and centrifuged for 7 minutes at 5400 rpm. The supernatant was aspirated and washing repeated one more time. Cell pellet was stored at −70° C. and used for glycome analysis.

Human Embryonic Stem Cell Lines (hESC)

Undifferentiated hESC Processes for generation of hESC lines from blastocyst stage in vitro fertilized excess human embryos have been described previously (e.g. Thomson et al., 1998). Two of the analysed cell lines in the present work were initially derived and cultured on mouse embryonic fibroblasts feeders (MEF; 12-13 pc fetuses of the ICR strain), and two on human foreskin fibroblast feeder cells (HFF; CRL-2429 ATCC, Mananas, USA). For the present studies all the lines were transferred on HFF feeder cells treated with mitomycin-C (1 g/ml; Sigma-Aldrich) and cultured in serum-free medium (Knockou™ D-MEM; Gibco® Cell culture systems, Invitrogen, Paisley, UK) supplemented with 2 mM L-Glutamin/Penicillin streptomycin (Sigma-Aldrich), 20% Knockout Serum Replacement (Gibco), 1× non-essential amino acids (Gibco), 0.1 mM β-mercaptoethanol (Gibco), 1×ITSF (Sigma-Aldrich) and 4 ng/ml bFGF (Sigma/Invitrogen).

Stage 2 differentiated hESC (embryoid bodies). To induce the formation of embryoid bodies (EB) the hESC colonies were first allowed to grow for 10-14 days whereafter the colonies were cut in small pieces and transferred on non-adherent Petri dishes to form suspension cultures. The formed EBs were cultured in suspension for the next 10 days in standard culture medium (see above) without bFGF.

Stage 3 differentiated hESC. For further differentiation EBs were transferred onto gelatin-coated (Sigma-Aldrich) adherent culture dishes in media consisting of DMEM/F12 mixture (Gibco) supplemented with ITS, Fibronectin (Sigma), L-glutamine and antibiotics. The attached cells were cultured for 10 days whereafter they were harvested.

Sample preparation. The cells were collected mechanically, washed, and stored frozen prior to glycan analysis.

### Experimental Procedures

Biological reagents. Bovine serum apotransferrin and fetuin were from Sigma (USA).

Glycan isolation. N-linked glycans were detached from cellular glycoproteins by F. meningosepticum N-glycosidase F digestion (Calbiochem, USA) essentially as described previously (Nyman et al., 1998), after which the released glycans were purified for analysis by solid-phase extraction methods, including ion exchange separation, and divided into sialylated and non-sialylated fractions.

MALDI-TOF mass spectrometry. MALDI-TOF mass spectrometry was performed with a Voyager-DE STR BioSpectrometry Workstation or a Bruker Ultraflex TOF/TOF instrument, essentially as described previously (Saarinen et al., 1999; Harvey et al., 1993). Relative molar abundancies of both neutral (Naven & Harvey, 1996) and sialylated (Papac et al., 1996) glycan components were assigned based on their relative signal intensities. The mass spectrometric fragmentation analysis was done with the Bruker Ultraflex TOF/TOF instrument according to manufacturer's instructions.

Sialic acid analysis. Sialic acids were released from sample glycoconjugates by mild propionic acid hydrolysis, reacted with 1,2-diamino-4,5-methylenedioxybenzene (DMB), and analyzed by reversed-phase high-performance liquid chromatography (HPLC) essentially as described previously (Ylönen et al., 2001).

### Results

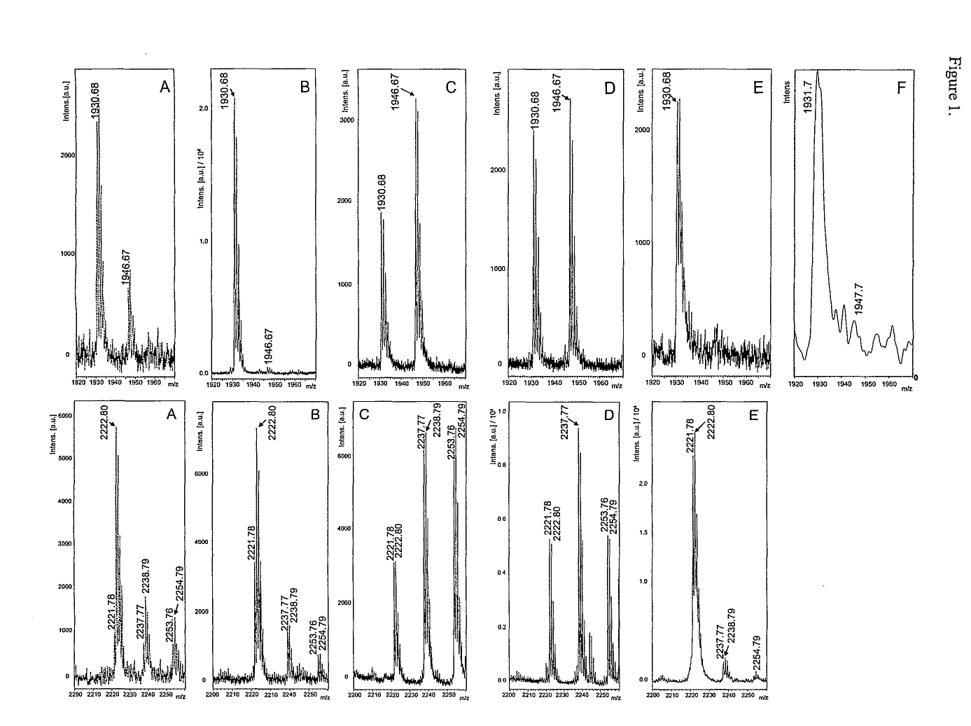

N-glycan analysis of stem cell samples. N-glycans from samples of various stem cell and differentiated cells, as well as from culture media and other biological reagents used in treatment of these samples, were isolated and fractionated into neutral and sialylated N-glycan fractions as described under Experimental procedures. In MALDI-TOF mass spectrometry of the sialylated N-glycan fractions, several glycan signals were detected in these samples that indicated the presence of N-glycolylneuraminic acid (Neu5Gc) in the N-glycans. As an example, FIG. 1 shows mass spectra of sialylated N-glycan fractions from stem cell samples (A. and B.), commercial cell culture media (C. and E.), and bovine serum glycoproteins (D. and F.). The glycan signals at m/z 1946 (upper panel), corresponding to the [M−H]⁻ ion of NeuGc₁Hex₅HexNAc₄, as well as m/z 2237 and m/z 2253 (lower panel), corresponding to the [M−H]⁻ ions of NeuGc₁NeuAc₁Hex₅HexNAc₄ and NeuGc₂Hex₅HexNAc₄, respectively, are indicative of the presence of N-glycolylneuraminic acid, i.e. a sialic acid residue with 16 Da larger mass than N-acetylneuraminic acid (Neu5Ac).

The indicative glycan signals and other signals proposed to correspond to Neu5Gc-containing glycan species are listed in Table 1, along with the mass spectrometric profiling results obtained from stem cell samples. CD133⁺ cells from human cord blood are representative of cord blood cell populations in the present example and other cell populations detected to contain similar Neu5Gc glycoconjugates included CD34⁺ and LIN⁻ cells from cord blood. Mesenchymal stem cells from human bone marrow are representative of mesenchymal stem cell lines in the present example and other mesenchymal stem cell lines detected to contain similar Neu5Gc glycoconjugates included cell lines derived from cord blood.

Mass spectrometric profiling results obtained from cell culture media and biological reagents are listed below. The indicative glycan signals and other signals proposed to correspond to Neu5Gc-containing glycan species in the studied reagents are listed in Table 6. The results indicate for the presence of Neu5Gc in the listed reagents.

Glycan profiling of reagents. N-glycans were liberated from reagents enzymatically by N-glycosidase F, purified and analysed by mass spectrometry. The results are summarized below.

### 1. Following Reagents Contained Detectable Amounts of N-Glycans:

Commercial BSA (bovine serum albumin)
Fetal bovine serum (FBS)
Transferrin, bovine serum
Horse serum
Monoclonal antibodies, including murine antibodies
Cell culture media

### 2. Reagents Containing N-Glycolyl Neuraminic Acid (NeuGc) or Acetyl-Groups (Ac):

Following lists present masses detected from mass spectra and their corresponding proposed monosaccharide compositions (exact calculated mass values are presented in Table 6).

### General Mono Saccharide Compositions of Common Animal N-Glycan Structures Comprising N-Glycolyl Neuraminic Acid (NeuGc) or Acetyl-Groups (Ac):

It is realized that various animal species produced large number of different proteins. The following general composition describes some useful major signals and the present invention is directed to especially analysis of these, preferably in context of serum/blood derived samples from mammals, preferably from horse and/or bovine. The invention is directed to the analysis of individual isolated proteins such as serum transferrin or an antibody, and preferably analysis of variation among the individual protein (depending on animal individual, condition of the animal, animal strain or species, for example). The invention is further and preferably directed to analysis of complex protein mixture such as animal tissue fractions such as blood fractions, more preferably serum fractions. The signals are especially useful as these are not commonly observed from human tissue or cell materials with contamination of animal material. As the glycans posses known antigenicity and other risks it is useful to analyse presence of these for example from therapeutic products such as therapeutic cell products or reagents aimed for production of these.

The invention is further directed to methods, especially research and development methods, such as product development methods, according to the invention including step of producing a qualitative and/or quantitative glycome analysis from cell culture directed materials or for development of these or producing a qualitative and/or quantitative glycome analysis from cells for revealing potential presence of or contamination by non-human protein material such as animal protein, such as a mammalian sialylated protein. It is realized that cell culture reagents can be produced by various cell culture methods producing non-human N-glycosylation such as preferably by non-mammalian or non-vertebrate cell systems preferably by plant, fungal, yeast or insect cells or engineered versions of these producing human similar glycomes, which have different biological activities need to be analysed and are analysed preferably by the methods according to the invention.

The general monosaccharide composition for characteristic major glycan signals (structures) to be analyzed in context of animal protein N-glycans, preferably from serum, (comprising NeuGc-glycans and/or O-acetyl structures such as O-acetylated sialic acids such as NeuNAc-OAc-glycans) is according to formula:

NeuGcₙ₁NeuNAcₙ₂Hexₙ₃HexNAcₙ₄dHexₙ₅Acₙ₆,

wherein n1 is an integer with values 0-5, preferably 0-4, more preferably 0-3, most preferably 0, 1 or 2;
n2 is an integer with values 0-5, preferably 0-4, more preferably 0-3, most preferably is 0, 1 or 2;
n3 is an integer having values from 1 to 8, more preferably values 1 or from 3-8, even more preferably values 1 or from 3-6, preferably from 3 to 6, most preferably 3, 5 or 6;
n4 is an integer having values from 2-7, more preferably 2-6, even more preferably 2-5 or 3-6 and most commonly preferably 3-5
n5 is an integer having values 0-3, and
n6 is an integer with values 0-4, preferred ranges further includes 0, 1 or 2, and 0, 1, 2 or 4.

It is realized that the protein composition may comprise multiple branched N-glycan increasing the amount of sialic acids and n3 and n4 with increasing amount of terminal N-acetyllactosamines. It is further realized that when the N-glycan comprise poly-n-acetyllactosamines the values of monosaccharide units in observable signals with n3 and n4 and optionally also number of sialic acid (n1 and n2 and possible acetylation there of n6), when branching is increased, and number of fucose (n5, increase in n5 is typically smaller than increase of N-acetyllactosamines), when fucosylation of N-acetyllactosamines is increased, can be and increased by numbers between about 1-10, more preferably the number of the monosaccharide units in compositions is increased with number between 1-5 or in case of common modest increase in N-acetyllactosamines the increase is 1-3.

The most preferred compositions were revealed to comprise monosaccharide compositions of common biantennary complex type N-glycans, and some unusual smaller variations thereof.

### Commercial BSA

The monosaccharide composition for characteristic glycan signals (structures) to be analyzed in context of commercial bovine serum transferring (comprising NeuGc-glycans) includes signals according formula:

NeuGcₙ₁NeuNAcₙ₂Hexₙ₃HexNAcₙ₄dHexₙ₅,

wherein n1 is 1 or 2;
n2 is 0, or 1;
n3 is an integer having values from 3-5;
n4 is an integer having values from 3-4 and
n5 is an integer having values 0 or 1, examples of preferred compositions are listed below.

The compositions are quite similar to bovine serum proteins but only part of glycans are included. Examples and most preferred signals to be analyzed from animal serum albumin, preferably bovine serum albumin, samples includes following mass signals and/or monosaccharide compositions:

| | |
|---|---|
| 1419 | NeuGcHex3HexNAc3 |
| 1581 | NeuGcHex4HexNAc3 |
| 1946 | NeuGcHex5HexNAc3 |
| 2237 | NeuGcNeuAcHex5HexNAc4 |
| 2253 | NeuGc2Hex5HexNAc4 |
| 2383 | NeuGcNeuAcHex5HexNAc4dHex |
| 2399 | NeuGc2Hex5HexNAc4dHex |
| 2528 | NeuGcNeuAc2Hex5HexNAc4 |
| 2544 | NeuGc2NeuAcHex5HexNAc4 |

### Commercial Antibody Preparations

O-acetylated NeuNAc residues were found. The present invention is in a preferred embodiment directed to analysis NeuAc-OAc comprising N-glycans from antibodies. The invention is especially directed to analysis of disialylated antibodies with following monosaccharide compositions. The compositions correspond to biantennary N-glycans comprising 1, 2, or 4 O-acetyl groups. It is realized that O-acetyl structures are likely antigenic and may affect also other biological activities of glycans such as interactions with sialic acid binding lectins, for example serum lectins in therapeutic or diagnostic applications. The invention is therefore directed to the analysis of Neu-OAc comprising glycans from commercial proteins such as antibodies.

The invention is especially directed to compositions:

NeuAc2Hex5HexNAc4Acn,

wherein n is an integer 1-4, preferably 1, 2, or 4, as shown below:

| | |
|---|---|
| 2263 | NeuAc2Hex5HexNAc4Ac (NeuAcHex4HexNAc5dHex2) |
| 2305 | NeuAc2Hex5HexNAc4Ac2 |
| 2389 | NeuAc2Hex5HexNAc4Ac4 |

### Transferrin, Bovine Serum

The monosaccharide composition for characteristic glycan signals (structures) to be analyzed in context of commercial bovine serum transferring (comprising NeuGc-glycans) includes signals according to formula:

NeuGcₙ₁NeuNAcₙ₂Hexₙ₃HexNAcₙ₄dHexₙ₅,

wherein n1 is 1 or 2;
n2 is 0, 1 or 2;
n3 is an integer having values from 3-6;
n4 is an integer having values from 3-4 and
n5 is an integer having values from 0-2, examples of preferred compositions are listed below.

The compositions are quite similar to commercial serum replacement shown below.

Examples and most preferred signals to be analyzed from animal serum transferrin, preferably bovine serum transferrin, samples includes following mass signals and/or monosaccharide compositions:

| | |
|---|---|
| 1419 | NeuGcHex3HexNAc3 |
| 1581 | NeuGcHex4HexNAc3 |
| 1784 | NeuGcHex4HexNAc4 |
| 1946 | NeuGcHex5HexNAc3 |
| 2092 | NeuGcHex5HexNAc4dHex |
| 2237 | NeuGcNeuNAcHex5HexNAc4 |
| 2253 | NeuGc2Hex5HexNAc4 |
| 2254 | NeuGcHex6HexNAc4dHex |
| 2383 | NeuGcNeuAcHex5HexNAc4dHex |
| 2399 | NeuGc2Hex5HexNAc4dHex |
| 2528 | NeuGcNeuAc2Hex5HexNAc4 |
| 2529 | NeuGcNeuAcHex5HexNAc4dHex2 |
| 2544 | NeuGc2NeuAcHex5HexNAc4 |
| 2545 | NeuGcNeuAcHex6HexNAc4dHex |

The invention further revealed that there is individual variation in the quantitative composition of individual animal glycoproteins, such as bovine serum proteins, preferably bovine serum transferrin. A preferred variable in to be determined is relative amount of sialylated and neutral glycans and/or relation of monosialylated and multiply sialylated glycans, preferably disialylated glycans; and/or the ratio of non-fucosylated glycans to mono- and or multiply fucosylated glycans; and or the ration of mono-fucosylated glycans to multiply fucosylated glycans. The quantitative composition means relative amounts of components of individual peaks, preferably measured as intensity of the peaks. The present invention is especially directed to determination of the quantitative composition of glycomes isolated from animal proteins and quantitative comparisons of the compositions.

### Horse Serum

The monosaccharide composition for characteristic glycan signals (structures) to be analyzed in context of horse serum type materials (comprising NeuGc-glycans) is according to formula:

NeuGcₙ₁NeuNAcₙ₂Hexₙ₃HexNAcₙ₄dHexₙ₅Acₙ₆,

wherein n1 is 0, 1 or 2;
n2 is 0, 1 or 2;
n3 is an integer having values from 3-5, preferably 3, 5 or 6;
n4 is an integer having values from 3-5,
n5 is an integer having values 0 or 1, and
n6 is an integer 0, 1 or 2.

| | |
|---|---|
| 2092 | NeuGcHex5HexNAc4dHex (NeuAcHex6HexNAc4) |
| 2237 | NeuGcNeuAcHex5HexNAc4 |
| 2238 | NeuGcHex5HexNAc4dHex2 (NeuAcHex6HexNAc4dHex) |
| 2254 | NeuGcHex6HexNAc4dHex (NeuAcHex7HexNAc4) |
| 2399 | NeuGc2Hex5HexNAc4dHex |

NeuGcₙ₁NeuNAcₙ₂Hexₙ₃HexNAcₙ₄dHexₙ₅Acₙ₆,

wherein n1 is 1 or 2;
n2 is 0, or 1;
n3 is an integer having values from 3-5, preferably 3, 5 or 6;
n4 is an integer having values from 3-5,
n5 is an integer having values 0 or 1, and
n6 is an integer 0; examples of preferred compositions are included above

| | |
|---|---|
| 1445 | NeuAcHex3HexNAc3Ac |
| 1972 | NeuAcHex5HexNAc4Ac |
| 2263 | NeuAc2Hex5HexNAc4Ac |
| 2305 | NeuAc2Hex5HexNAc4Ac2 |
| 2321 | NeuAcHex5HexNAc5dHexAc |
| 2409 | NeuAc2Hex5HexNAc4dHexAc |
| 2451 | NeuAc2Hex5HexNAc4dHexAc2 |
| 2483 | NeuAcHex6HexNAc5dHexAc |
| 2467 | NeuAcHex5HexNAc5dHex2Ac |

The preferred acetylated sequences analysed to correspond to O-acetylated sialic acids (NeuAc) are according to the formula:

(NeuGcₙ₁)NeuNAcₙ₂Hexₙ₃HexNAcₙ₄dHexₙ₅Acₙ₆,

wherein n1 is 0, the sequences comprise practically all NeuNAc;
n2 is 0, 1;
n3 is an integer having values from 3-6, preferably 3, 5 or 6;
n4 is an integer having values from 3-5,
n5 is an integer having values 0, 1 or 2, and
n6 is an integer 0, 1 or 2; examples of preferred compositions are included above.

### 3. Cell Culture Media Analysed:

### Commercial Serum Replacement Cell Culture Media

The monosaccharide composition for characteristic glycan signals (structures) to be analyzed in context of commercial serum replacement cell culture media (comprising NeuGc-glycans) includes signals according to formula:

NeuGcₙ₁NeuNAcₙ₂Hexₙ₃HexNAcₙ₄dHexₙ₅,

wherein n1 is 0, 1 or 2, preferably 1 or 2;
n2 is 0, or 1;
n3 is an integer having values from 3-7, preferably 3-6;
n4 is an integer having values from 3-5 and
n5 is an integer having values from 0-3.

| | |
|---|---|
| 1419 | NeuGcHex3HexNAc3 |
| 1581 | NeuGcHex4HexNAc3 |
| 1727 | NeuGcHex4HexNAc3dHex |
| 1743 | NeuGcHex5HexNAc3 |
| 1784 | NeuGcHex4HexNAc4 |
| 1946 | NeuGcHex5HexNAc3 |
| 2092 | NeuGcHex5HexNAc4dHex |
| 2237 | NeuGcNeuAcHex5HexNAc4 |
| 2238 | NeuGcHex5HexNAc4dHex2 |
| 2253 | NeuGc2Hex5HexNAc4 |
| 2254 | NeuGcHex6HexNAc4dHex |
| 2383 | NeuGcNeuAcHex5HexNAc4dHex |
| 2384 | NeuGcHex5HexNAc4dHex3 |
| 2399 | NeuGc2Hex5HexNAc4dHex |
| 2528 | NeuGcNeuAc2Hex5HexNAc4 |
| 2529 | NeuGcNeuAcHex5HexNAc4dHex2 |
| 2544 | NeuGc2NeuAcHex5HexNAc4 |
| 2545 | NeuGcNeuAcHex6HexNAc4dHex |
| 2560 | NeuGc3Hex5HexNAc4 |
| 2602 | NeuGcNeuAcHex6HexNAc5 |
| 2603 | NeuGcHex6HexNAc5dHex2 (NeuAcHex7HexNAc5dHex) |

### FBS-Containing Cell Culture Media

The monosaccharide composition for characteristic glycan signals (structures) to be analyzed in context of fetal bovine serum is according to formula:

NeuGcₙ₁NeuNAcₙ₂Hexₙ₃HexNAcₙ₄dHexₙ₅Acₙ₆,

wherein n1 is 0, 1 or 2;
n2 is 0, 1 or 2;
n3 is an integer being 1, or 4-6;
n4 is an integer having values from 2-6 and
n5 is an integer having values 0, 1 or 2.
n6 is an integer 0, or 1; examples of preferred compositions are included above.

The invention is especially directed to analysis of presence of unusual signals at m/z 1038, NeuGcHexHexNAc2dHex, and 1329 NeuGcNeuAcHexHexNAc2dHex, the invention is further directed to the analysis of such structures from bovine serum, especially from FBS, preferably by specific glycosidase reagents, and/or fragmentation mass spectrometry and/or NMR-spectrometry.

| | |
|---|---|
| 1038 | NeuGcHexHexNAc2dHex |
| 1329 | NeuGcNeuAcHexHexNAc2dHex |
| 1727 | NeuGcHex4HexNAc3dHex |
| 1946 | NeuGcHex5HexNAc3 |
| 2092 | NeuGcHex5HexNAc4dHex |
| 2237 | NeuGcNeuAcHex5HexNAc4 |
| 2238 | NeuGcHex5HexNAc4dHex2 |
| 2253 | NeuGc2Hex5HexNAc4 |
| 2254 | NeuGcHex6HexNAc4dHex |
| 2366 | NeuGcNeuAc2Hex4HexNAc4 |
| 2383 | NeuGcNeuAcHex5HexNAc4dHex |
| 2409 | NeuAc2Hex5HexNAc4dHexAc |
| 2528 | NeuGcNeuAc2Hex5HexNAc4 |
| 2529 | NeuGcNeuAcHex5HexNAc4dHex2 |
| 2544 | NeuGc2NeuAcHex5HexNAc4 |
| 2602 | NeuGcNeuAcHex6HexNAc5 |
| 2618 | NeuGc2Hex6HexNAc5 |
| 2674 | NeuGcNeuAc2Hex5HexNAc4dHex |
| 2893 | NeuGcNeuAc2Hex6HexNAc5 |

### Horse Serum Containing Cell Culture Media

The monosaccharide composition for characteristic glycan signals (structures) to be analyzed in context of horse serum comprising cell culture media (comprising NeuGc-glycans and/or NeuNAc-OAc-glycans) is according to formula:

NeuGcₙ₁NeuNAcₙ₂Hexₙ₃HexNAcₙ₄dHexₙ₅Acₙ₆,

wherein n1 is 0, 1 or 2;
n2 is 0, 1 or 2;
n3 is an integer having values from 3-6, preferably 3, 5 or 6;
n4 is an integer having values from 3-5,
n5 is an integer having values 0 or 1, and
n6 is an integer 0, 1 or 2.

| | |
|---|---|
| 1581 | NeuGcHex4HexNAc3 |
| 1711 | NeuGcHex3HexNAc3dHex2 |
| 1727 | NeuGcHex4HexNAc3 |
| 1873 | NeuGcHex4HexNAc3dHex2 |
| 1946 | NeuGcHex5HexNAc3 |
| 2092 | NeuGcHex5HexNAc4dHex |
| 2237 | NeuGcNeuAcHex5HexNAc4 |
| 2238 | NeuGcHex5HexNAc4dHex2 |
| 2310 | NeuGcNeuAcHex4HexNAc3dHex3 |
| 2366 | NeuGcNeuAc2Hex4HexNAc4 |
| 2383 | NeuGcNeuAcHex5HexNAc4dHex |
| 2384 | NeuGcHex5HexNAc4dHex3 |
| 2399 | NeuGc2Hex5HexNAc4dHex |

The preferred subgroup of NeuGc comprising glycans includes

NeuGcₙ₁NeuNAcₙ₂Hexₙ₃HexNAcₙ₄dHexₙ₅Acₙ₆,

wherein n1 is 1 or 2;
n2 is 0, 1 or 2;
n3 is an integer having values from 3-5;
n4 is an integer having values 3 or 4,
n5 is an integer having values 0-4, and
n6 is an integer 0.

It is realized that the horse serum comprising medium contained signals 2310, 2366, 2383, and 2384 in addition to the horse serum signals listed above and lacked the signal at m/z 2254. These are considered as characteristic signals/components differentiating between animal protein materials, especially in comparison between horse derived materials.

| | |
|---|---|
| 1972 | NeuAcHex5HexNAc4Ac |
| 2263 | NeuAc2Hex5HexNAc4Ac |
| 2305 | NeuAc2Hex5HexNAc4Ac2 |
| 2321 | NeuAcHex5HexNAc5dHexAc |
| 2409 | NeuAc2Hex5HexNAc4dHexAc |
| 2483 | NeuAcHex6HexNAc5dHexAc |

It is notable that the horse serum derived cell culture media contained more NeuGc comprising glycan structures and less NeuNAc-OAc structures lacking peaks at m/z 1445 2451 and 2467 in comparison to the horse serum sample above. These are considered as characteristic signals/components differentiating between animal protein materials, especially in comparison between horse derived materials.

The invention is directed to analysis of variation of animal derived cell culture materials such as serum proteins used for cell culture and use of the monosaccharide compositions and/or the characteristics signals for analysis of differences between animal protein materials, especially animal derived cell culture materials or materials to best tested for suitability for such materials. The invention is directed to variation related to individual animals within the same species and being the source of, or producing, the sample materials and analysis of variations between animal species. In a preferred embodiment the invention is directed to recognition of the source (tissue type such as serum, individual animal, animal strain or animal species) of the protein by analysis of expressed glycans.

In a preferred embodiment the invention is directed to the analysis of, preferably analysis of presence or absence or level of, O-acetylated sialic acid in the sample material and/or the analysis of, preferably analysis of presence or absence or level of, NeuGc sialic acid and/or presence of various signals/monosaccharide compositions/structures differentiating animal protein samples. The invention is especially directed to simultaneous analysis of O-acetylated sialic acid and NeuGc, preferably by specific binding molecules such as specific binding proteins or more preferably by physical methods such as NMR and/or mass spectrometry, most preferably MALDI-TOF mass spectrometry.

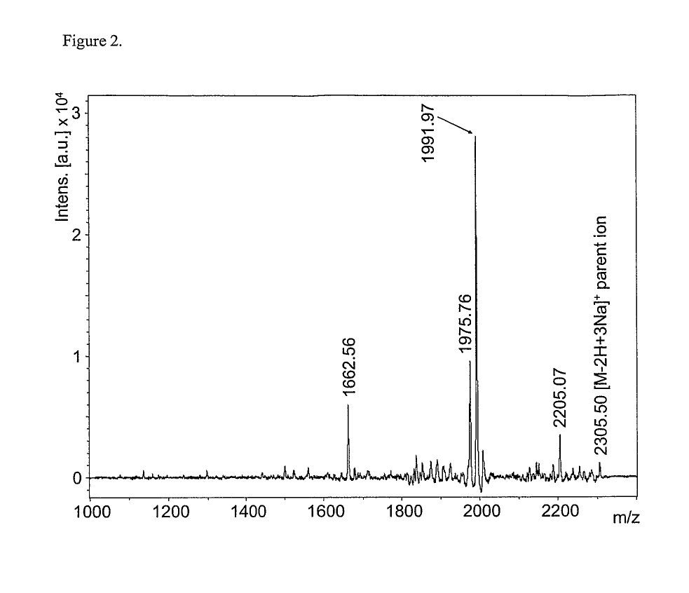

Fragmentation mass spectrometry. A sample of sialylated N-glycans isolated from cord blood CD133⁺ cells was subjected to mass spectrometric fragmentation analysis. Two different sodium adduct signals at m/z 2261 [M+Na]⁺ and 2305 [M−2H+3Na]⁺ were selected for fragmentation. The fragmentation spectrum of the [M−2H+3Na]⁺ ion at m/z 2305.50 (calc. m/z 2305.73) together with the proposed fragment ions is depicted in FIG. 2. The glycan signals at m/z 1975.76, corresponding to the ion [NeuAcHex₅HexNAc₄-H+2Na]⁺ (calc. m/z 1976.66), at m/z 1991.97, corresponding to the ion [NeuGcHex₅HexNAc₄-H+2Na]⁺ (calc. m/z 1992.65), and at m/z 1662.56, corresponding to the ion [Hex₅HexNAc₄+Na]⁺ (calc. m/z 1663.58), indicate the presence of one N-glycolyl neuraminic acid sodium salt residue (M_{NeuGc-H+Na}=329) and one N-acetyl neuraminic acid sodium salt residue (M_{NeuAc-H+Na}=313) in the original N-glycan ion. The fragmentation spectrum of the [M+Na]⁺ ion at m/z 2261.86 (calc. m/z 2261.77), yielded a similar result, and the resulting fragment signals at m/z 1954.45, corresponding to the ion [NeuAcHex₅HexNAc₄+Na]⁺ (calc. m/z 1954.68), at m/z 1969.93, corresponding to the ion [NeuGcHex₅HexNAc₄+Na]⁺ (calc. m/z 1970.67), and at m/z 1664.82, corresponding to the ion [Hex₅HexNAc₄+Na]⁺ (calc. m/z 1663.58), similarly indicate the presence of one N-glycolyl neuraminic acid residue (M_{NeuGc}=307) and one N-acetyl neuraminic acid residue (M_{NeuAc}=291) in the original N-glycan ion. In conclusion, the fragmentation analysis indicates that in the positive ion mode spectrum the glycan signals at m/z 2261 and 2305 correspond to the [M+Na]⁺ and [M−2H+3Na]⁺ ions of NeuAcNeuGcHex₅HexNAc₄, respectively, and in the negative ion mode spectrum the glycan signal at m/z 2237 corresponds to the [M−H]⁻ ion of NeuAcNeuGcHex₅HexNAc₄.

Sialic acid analysis. As described above, mass spectrometric profiling analyses indicated the presence of Neu5Gc in various cell samples and biological reagents. The sialic acid composition of commercial bovine serum transferrin was analyzed as described under Experimental procedures. The analysis indicated that the sample contained Neu5Gc and Neu5Ac in an approximate ratio of 50:50. The result was practically similar to mass spectrometric profiling that indicated that sialylated N-glycans isolated from the same sample contained Neu5Gc and Neu5Ac in a ratio of 53:47, as calculated from the proposed monosaccharide compositions of the detected glycan signals and their relative signal intensities. However, this Neu5Gc:Neu5Ac composition was significantly different from earlier reports of bovine serum transferrin sialic acid analysis results (e.g. 64:36, Rohrer et al., 1998), indicating that individual glycoprotein batches can differ from each other with regard to their sialic acid composition.

### Example 2

### Sialic Acid Linkage Analysis of Cord Blood Mononuclear Cell and Leukocyte Populations, and Bone Marrow Mesenchymal Stem Cells

### Experimental Procedures

N-glycan isolation from cord blood cell populations. Human cord blood mononuclear cells were isolated and divided into CD133⁺ and CD133⁻ cell populations as described above. N-linked glycans were detached from cellular glycoproteins and analyzed by mass spectrometry as described above.

α2,3-sialidase digestion. Sialylated N-glycans were treated with S. pneumoniae α2,3-sialidase (Glyko, UK) essentially as described previously (Saarinen et al., 1999). The sialic acid linkage specificity was controlled with synthetic oligosaccharides in parallel control reactions, and it was confirmed that in the reaction conditions the enzyme hydrolyzed α2,3-linked but not α2,6-linked sialic acids. After the enzymatic reaction, the glycans were purified and divided into sialylated and non-sialylated fractions and analyzed by mass spectrometry as described above.

Lectin stainings. FITC-labeled Maackia amurensis agglutinin (MAA) was purchased from EY Laboratories (USA) and FITC-labeled Sambucus nigra agglutinin (SNA) was purchased from Vector Laboratories (UK). Bone marrow derived mesenchymal stem cell lines were cultured as described above. After culturing, cells were rinsed 5 times with PBS (10 mM sodium phosphate, pH 7.2, 140 mM NaCl) and fixed with 4% PBS-buffered paraformaldehyde pH 7.2 at room temperature (RT) for 10 minutes. After fixation, cells were washed 3 times with PBS and non-specific binding sites were blocked with 3% HSA-PBS (FRC Blood Service, Finland) or 3% BSA-PBS (>99% pure BSA, Sigma) for 30 minutes at RT. According to manufacturers' instructions cells were washed twice with PBS, TBS (20 mM Tris-HCl, pH 7.5, 150 mM NaCl, 10 mM CaCl₂) or HEPES-buffer (10 mM HEPES, pH 7.5, 150 mM NaCl) before lectin incubation. FITC-labeled lectins were diluted in 1% HSA or 1% BSA in buffer and incubated with the cells for 60 minutes at RT in the dark. Furthermore, cells were washed 3 times 10 minutes with PBS/TBS/HEPES and mounted in Vectashield mounting medium containing DAPI-stain (Vector Laboratories, UK). Lectin stainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

### Results

Mass spectrometric analysis of cord blood CD133⁺ and CD133⁻ cell N-glycans. Sialylated N-glycans were isolated from cord blood CD133⁺ and CD133⁻ cell fractions and analyzed by MALDI-TOF mass spectrometry as described under Experimental procedures, allowing for relative quantitation of individual N-glycan signals.

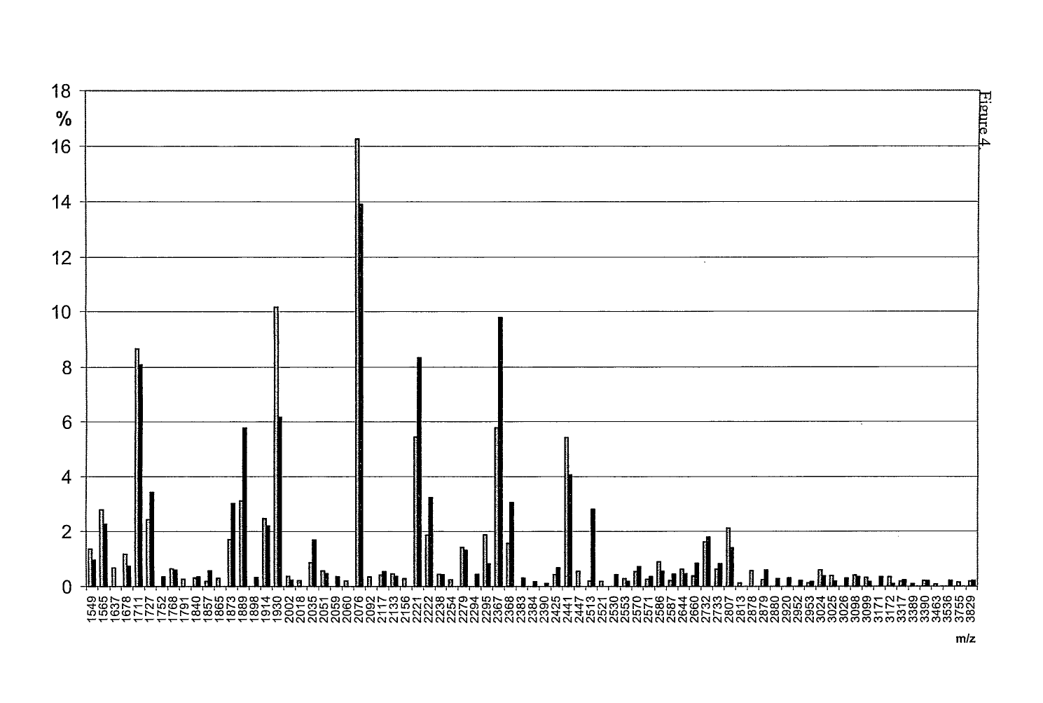

Cord blood CD133⁺ and CD133⁻ cell N-glycans are differentially α2,3-sialylated. Sialylated N-glycans from cord blood CD133⁺ and CD133⁻ cells were treated with α2,3-sialidase, after which the resulting glycans were divided into sialylated and non-sialylated fractions, as described under Experimental procedures. Both α2,3-sialidase resistant and sensitive sialylated N-glycans were observed, i.e. after the sialidase treatment sialylated glycans were observed in the sialylated N-glycan fraction and desialylated glycans were observed in the neutral N-glycan fraction. The results indicate that cord blood CD133⁺ and CD133⁻ cells are differentially α2,3-sialylated. For example, after α2,3-sialidase treatment the relative proportions of monosialylated (SA₁) glycan signal at m/z 2076, corresponding to the [M−H]⁻ ion of NeuAc₁Hex₅HexNAc₄dHex₁, and the disialylated (SA₂) glycan signal at m/z 2367, corresponding to the [M−H]⁻ ion of NeuAc₂Hex₅HexNAc₄dHex₁, indicate that α2,3-sialidase resistant disialylated N-glycans are relatively more abundant in CD133⁻ than in CD133⁺ cells, when compared to α2,3-sialidase resistant monosialylated N-glycans (FIG. 4). It is concluded that N-glycan α2,3-sialylation in relation to other sialic acid linkages including especially α2,6-sialylation, is more abundant in cord blood CD133⁺ cells than in CD133⁻ cells.

In cord blood CD133⁻ cells, several sialylated N-glycans were observed that were resistant to α2,3-sialidase treatment, i.e. neutral glycans were not observed that would correspond to the desialylated forms of the original sialylated glycans. The results revealing differential α2,3-sialylation of individual N-glycan structures between cord blood CD133⁺ and CD133⁻ cells are presented in Table 2. The present results indicate that N-glycan α2,3-sialylation in relation to other sialic acid linkages is more abundant in cord blood CD133⁺ cells than in CD133⁻ cells.

Lectin binding analysis of mesenchymal stem cells. As described under Experimental procedures, bone marrow derived mesenchymal stem cells were analyzed for the presence of ligands of α2,3-linked sialic acid specific (MAA) and α2,6-linked sialic acid specific (SNA) lectins on their surface. It was revealed that MAA bound strongly to the cells whereas SNA bound weakly, indicating that in the cell culture conditions, the cells had significantly more α2,3-linked than α2,6-linked sialic acids on their surface glycoconjugates.

### Example 3

### Enzymatic Modification of Cell Surface Glycan Structures

### Experimental Procedures

Enzymatic modifications. Sialyltransferase reaction: Human cord blood mononuclear cells (3×10⁶ cells) were modified with 60 mU α2,3-(N)-sialyltransferase (rat, recombinant in S. frugiperda, Calbiochem), 1.6 μmol CMP-Neu5Ac in 50 mM sodium 3-morpholinopropanesulfonic acid (MOPS) buffer pH 7.4, 150 mM NaCl at total volume of 100 μl for up to 12 hours. Fucosyltransferase reaction: Human cord blood mononuclear cells (3×10⁶ cells) were modified with 4 mU α1,3-fucosyltransferase VI (human, recombinant in S. frugiperda, Calbiochem), 1 μmol GDP-Fuc in 50 mM MOPS buffer pH 7.2, 150 mM NaCl at total volume of 100 μl for up to 3 hours. Broad-range sialidase reaction: Human cord blood mononuclear cells (3×10⁶ cells) were modified with 5 mU sialidase (A. ureafaciens, Glyko, UK) in 50 mM sodium acetate buffer pH 5.5, 150 mM NaCl at total volume of 100 μl for up to 12 hours. α2,3-specific sialidase reaction: Cells were modified with α2,3-sialidase (S. pneumoniae, recombinant in E. coli) in 50 mM sodium acetate buffer pH 5.5, 150 mM NaCl at total volume of 100 μl. Sequential enzymatic modifications: Between sequential reactions cells were pelleted with centrifugation and supernatant was discarded, after which the next modification enzyme in appropriate buffer and substrate solution was applied to the cells as described above. Washing procedure: After modification, cells were washed with phosphate buffered saline.

Glycan analysis. After washing the cells, total cellular glycoproteins were subjected to N-glycosidase digestion, and sialylated and neutral N-glycans isolated and analyzed with mass spectrometry as described above. For O-glycan analysis, the glycoproteins were subjected to reducing alkaline β-elimination essentially as described previously (Nyman et al., 1998), after which sialylated and neutral glycan alditol fractions were isolated and analyzed with mass spectrometry as described above.

### Glycans Remodelled by Glycosyltransferases/Glycosyltransferase

The present invention is further directed to special glycan controlled reagent produced by process including steps
1) Optionally partially depleting glycan structure as described by the invention, the partially depleted glycan structure may be also a non-animal structure as described for group 2 of glycan depleted reagents or a glycosylated protein from a prokaryote.
2) Transferring an acceptable or non-harmful glycan to glycan of reagent. Such process is known as glycoprotein remodelling for certain therapeutic proteins. The inventors revealed that there is a need for a remodelling process for specific reagents present in cell culture processes.
Furthermore the inventors were able to show glycan depletion and/or remodelling of large protein mixtures even for total serum involving numerous factors potentially inhibiting transfer reactions.

### Results

Sialidase digestion. Upon broad-range sialidase catalyzed desialylation of living cord blood mononuclear cells, sialylated N-glycan structures as well as O-glycan structures (data not shown) were desialylated, as indicated by increase in relative amounts of corresponding neutral N-glycan structures, for example Hex₆HexNAc₃, Hex₅HexNAc₄dHex₀₋₂, and Hex₆HexNAc₅dHex₀₋₁ monosaccharide compositions (Table 5). In general, a shift in glycosylation profiles towards glycan structures with less sialic acid residues was observed in sialylated N-glycan analyses upon broad-range sialidase treatment. The shift in glycan profiles of the cells upon the reaction served as an effective means to characterize the reaction results. It is concluded that the resulting modified cells contained less sialic acid residues and more terminal galactose residues at their surface after the reaction.

α2,3-specific sialidase digestion. Similarly, upon α2,3-specific sialidase catalyzed desialylation of living mononuclear cells, sialylated N-glycan structures were desialylated, as indicated by increase in relative amounts of corresponding neutral N-glycan structures (data not shown). In general, a shift in glycosylation profiles towards glycan structures with less sialic acid residues was observed in sialylated N-glycan analyses upon α2,3-specific sialidase treatment. The shift in glycan profiles of the cells upon the reaction served as an effective means to characterize the reaction results. It is concluded that the resulting modified cells contained less α2,3-linked sialic acid residues and more terminal galactose residues at their surface after the reaction.

Sialyltransferase reaction. Upon α2,3-sialyltransferase catalyzed sialylation of living cord blood mononuclear cells, numerous neutral (Table 5) and sialylated N-glycan (Table 4) structures as well as O-glycan structures (data not shown) were sialylated, as indicated by decrease in relative amounts of neutral N-glycan structures (Hex₅HexNAc₄dHex₀₋₃ and Hex₆HexNAc₅dHex₀₋₂ monosaccharide compositions in Table 5) and increase in the corresponding sialylated structures (for example the NeuAc₂Hex₅HexNAc₄dHex₁ glycan in Table 4). In general, a shift in glycosylation profiles towards glycan structures with more sialic acid residues was observed both in N-glycan and O-glycan analyses. It is concluded that the resulting modified cells contained more α2,3-linked sialic acid residues and less terminal galactose residues at their surface after the reaction.

Fucosyltransferase reaction. Upon α1,3-fucosyltransferase catalyzed fucosylation of living cord blood mononuclear cells, numerous neutral (Table 5) and sialylated N-glycan structures as well as O-glycan structures (see below) were fucosylated, as indicated by decrease in relative amounts of nonfucosylated glycan structures (without dHex in the proposed monosaccharide compositions) and increase in the corresponding fucosylated structures (with n_{dHex}>0 in the proposed monosaccharide compositions). For example, before fucosylation O-glycan alditol signals at m/z 773, corresponding to the [M+Na]⁺ ion of Hex₂HexNAc₂ alditol, and at m/z 919, corresponding to the [M+Na]⁺ ion of Hex₂HexNAc₂dHex₁ alditol, were observed in approximate relative proportions 9:1, respectively (data not shown). After fucosylation, the approximate relative proportions of the signals were 3:1, indicating that significant fucosylation of neutral O-glycans had occurred. Some fucosylated N-glycan structures were even observed after the reaction that had not been observed in the original cells, for example neutral N-glycans with proposed structures Hex₆HexNAc₅dHex₁ and Hex₆HexNAc₅dHex₂ (Table 5), indicating that in α1,3-fucosyltransferase reaction the cell surface of living cells can be modified with increased amounts or extraordinary structure types of fucosylated glycans, especially terminal Lewis x epitopes in protein-linked N-glycans as well as in O-glycans.

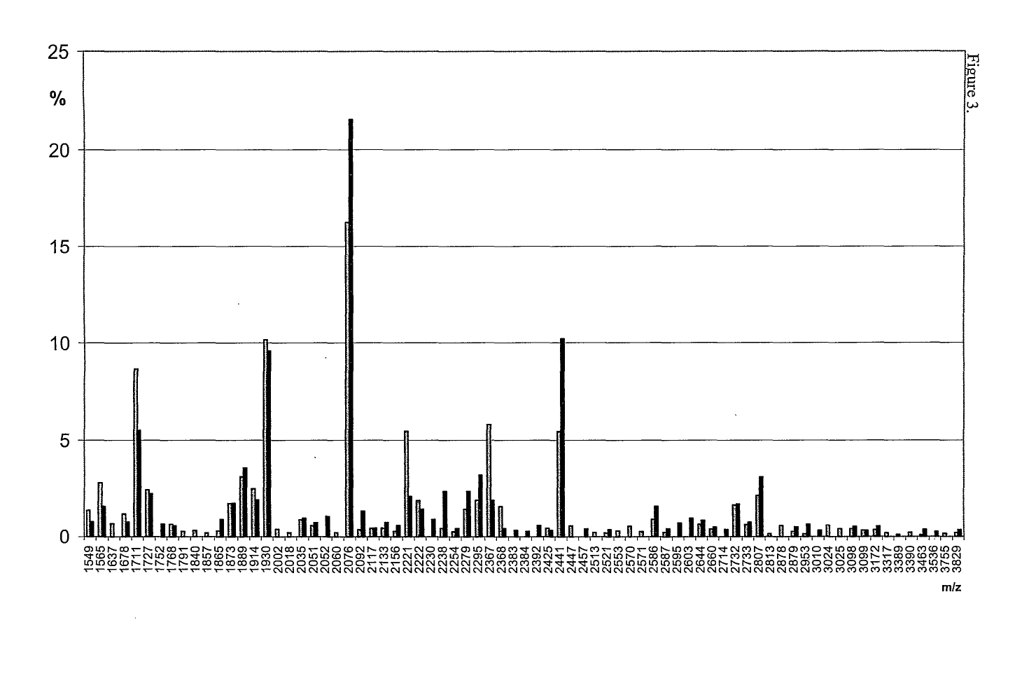

Sialidase digestion followed by sialyltransferase reaction. Cord blood mononuclear cells were subjected to broad-range sialidase reaction, after which α2,3-sialyltransferase and CMP-Neu5Ac were added to the same reaction, as described under Experimental procedures. The effects of this reaction sequence on the N-glycan profiles of the cells are described in FIG. 3. The sialylated N-glycan profile was also analyzed between the reaction steps, and the result clearly indicated that sialic acids were first removed from the sialylated N-glycans (indicated for example by appearance of increased amounts of neutral N-glycans), and then replaced by α2,3-linked sialic acid residues (indicated for example by disappearance of the newly formed neutral N-glycans; data not shown). It is concluded that the resulting modified cells contained more α2,3-linked sialic acid residues after the reaction.

Sialyltransferase reaction followed by fucosyltransferase reaction. Cord blood mononuclear cells were subjected to α2,3-sialyltransferase reaction, after which α1,3-fucosyltransferase and GDP-fucose were added to the same reaction, as described under Experimental procedures. The effects of this reaction sequence on the sialylated N-glycan profiles of the cells are described in FIG. 4. The results show that a major part of the glycan signals (detailed in Table 3) have undergone changes in their relative intensities, indicating that a major part of the sialylated N-glycans present in the cells were substrates of the enzymes. It was also clear that the combination of the enzymatic reaction steps resulted in different result than either one of the reaction steps alone.

Different from the α1,3-fucosyltransferase reaction described above, sialylation before fucosylation apparently sialylated the neutral fucosyltransferase acceptor glycan structures present on cord blood mononuclear cell surfaces, resulting in no detectable formation of the neutral fucosylated N-glycan structures that had emerged after α1,3-fucosyltransferase reaction alone (discussed above; Table 5).

Glycosyltransferase-derived glycan structures. We detected that glycosylated glycosyltransferase enzymes can contaminate cells in modification reactions. For example, when cells were incubated with recombinant fucosyltransferase or sialyltransferase enzymes produced in S. frugiperda cells, N-glycosidase and mass spectrometric analysis of cellular and/or cell-associated glycoproteins resulted in detection of an abundant neutral N-glycan signal at m/z 1079, corresponding to [M+Na]⁺ ion of Hex₃HexNAc₂dHex₁ glycan component (calc. m/z 1079.38). Typically, in recombinant glycosyltransferase treated cells, this glycan signal was more abundant than or at least comparable to the cells' own glycan signals, indicating that insect-derived glycoconjugates are a very potent contaminant associated with recombinant glycan-modified enzymes produced in insect cells. Moreover, this glycan contamination persisted even after washing of the cells, indicating that the insect-type glycoconjugate corresponding to or associated with the glycosyltransferase enzymes has affinity towards cells or has tendency to resist washing from cells. To confirm the origin of the glycan signal, we analyzed glycan contents of commercial recombinant fucosyltransferase and sialyltransferase enzyme preparations and found that the m/z 1079 glycan signal was a major N-glycan signal associated with these enzymes. Corresponding N-glycan structures, e.g. Manα3(Manα6)Manβ4GlcNAc(Fucα3/6)GlcNAc(β-N-Asn), have been described previously from glycoproteins produced in S. frugiperda cells (Staudacher et al., 1992; Kretzchmar et al., 1994; Kubelka et al., 1994; Altmann et al., 1999). As described in the literature, these glycan structures, as well as other glycan structures potentially contaminating cells treated with recombinant or purified enzymes, especially insect-derived products, are potentially immunogenic in humans and/or otherwise harmful to the use of the modified cells. It is concluded that glycan-modifying enzymes must be carefully selected for modification of human cells, especially for clinical use, not to contain immunogenic glycan epitopes, non-human glycan structures, and/or other glycan structures potentially having unwanted biological effects.

### Example 4

### Analysis of Stability and Cultivation Properties of Glycosidase or Glycosyltransferase Modified Cells

Stability and cultivation properties of neuraminidase and glycosyltransferase (sialyltransferase and fucosyltransferase) modified cells from previous example were analyzed in CFU cell culture assay and viability assay as described in (Kekarainen et al BMC Cell Biol (2006) 7, 30).

The invention revealed that the modified cord blood mononuclear cells with quantitatively reduced sialic acid levels gave in CFU cell culture assay higher colony counts. The invention is especially directed to the use of the desialylated hematopoietic cells for cultivation of blood cell populations, especially for cultivation of hematopoietic cells (Table 7).

### Example 5

### Linkage Specific Desialylation

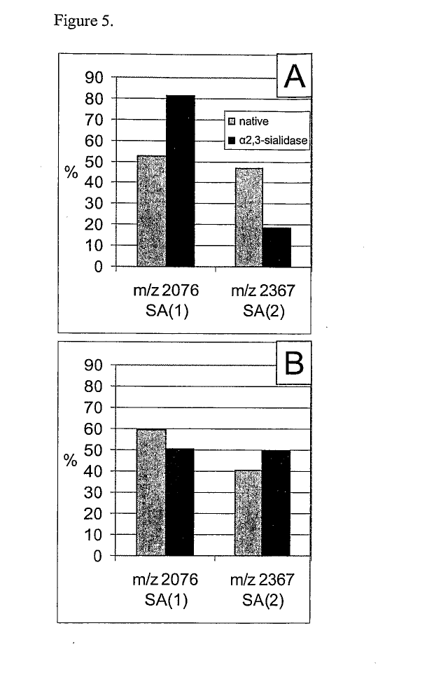

Hematopoietic stem cell fractions from cord blood are treated with α3-linkage specific sialidase as indicated in EXAMPLE 3. Differences in desialylation by linkage specific sialidase can be observed by comparing signals of monosialylated and disialyted glycans as shown in FIG. 5.

### Example 6

### Glycan Controlled Enzyme

Glycosylation of commercial sialyl- or fucosyltransferase (Calbiochem CA) enzyme produced in insect cells is controlled by releasing the glycans, purifying the glycans and MALDI-TOF mass spectrometry (WO publication by the inventors, filed 11.7.2005). Potentially allergenic insect type glycans are observed and released by exoglycosidase enzymes as described for the insect glycans such as α-mannosidase, β-mannosidase α3- or α3/α6-fucosidases and hexosaminidase (such as Jack bean hexosaminidase), (WO publication by the inventors, filed 11 Jul. 2005).

Glycosyltransferase-derived glycan structures. We detected that glycosylated glycosyltransferase enzymes can contaminate cells in modification reactions. For example, when cells were incubated with recombinant fucosyltransferase or sialyltransferase enzymes produced in S. frugiperda cells, N-glycosidase and mass spectrometric analysis of cellular and/or cell-associated glycoproteins resulted in detection of an abundant neutral N-glycan signal at m/z 1079, corresponding to [M+Na]⁺ ion of Hex₃HexNAc₂dHex₁ glycan component (calc. m/z 1079.38). Typically, in recombinant glycosyltransferase treated cells, this glycan signal was more abundant than or at least comparable to the cells' own glycan signals, indicating that insect-derived glycoconjugates are a very potent contaminant associated with recombinant glycan-modified enzymes produced in insect cells. Moreover, this glycan contamination persisted even after washing of the cells, indicating that the insect-type glycoconjugate corresponding to or associated with the glycosyltransferase enzymes has affinity towards cells or has tendency to resist washing from cells. To confirm the origin of the glycan signal, we analyzed glycan contents of commercial recombinant fucosyltransferase and sialyltransferase enzyme preparations and found that the m/z 1079 glycan signal was a major N-glycan signal associated with these enzymes. Corresponding N-glycan structures, e.g. Manα3(Manα6)Manβ4GlcNAc(Fucα3/6)GlcNAc(β-N-Asn), have been described previously from glycoproteins produced in S. frugiperda cells (Staudacher et al., 1992; Kretzchmar et al., 1994; Kubelka et al., 1994; Altmann et al., 1999). As described in the literature, these glycan structures, as well as other glycan structures potentially contaminating cells treated with recombinant or purified enzymes, especially insect-derived products, are potentially immunogenic in humans and/or otherwise harmful to the use of the modified cells. It is concluded that glycan-modifying enzymes must be carefully selected for modification of human cells, especially for clinical use, not to contain immunogenic glycan epitopes, non-human glycan structures, and/or other glycan structures potentially having unwanted biological effects or in a preferred embodiment the glycan structures are removed or degraded to non-harmful ones.

### Example 7

### Use of Tagged Enzyme

Neuraminidase or sialyltransferase is biotinylated as described in catalog of Pierce. Biotinylated neuraminidase or sialyltransferase enzyme is incubated with mononuclear blood cells to remodel the cellular glycosylation as described in the invention. The enzyme is removed by (strept)avidin magnetic beads (e.g. Miltenyi or Dynal) optionally with presence of neuraminic acid and or sialyltransferase acceptor (N-acetyllactosamine or lactose).

### Example 8

### Modification of BM-MSC Cells

### Materials and Methods

### Cells

Bone marrow (BM)-derived mesenchymal stem cells (MSCs) were obtained as described by Leskelä et al. (2003). After initial culture establishment, BM-MSCs (<passage 10) were cultured in a humidified 5% CO₂ atmosphere at +37° C. in Minimum Essential Alpha-Medium (αMEM) (Gibco) supplemented with 10% FCS, 20 mM Hepes, 10 ml/l penicillin/streptomycin and 2 mM L-glutamine.

### α2,3-Sialyltransferase Enzymatic Modification

Cell culture media was collected for further analysis before the detachment. For the enzymatic modifications, BM-MSCs at 70-80% confluency were detached with PBS+2 mM Na-EDTA (Versene) for 30 min at +37° C. The detached cells were calculated in Bürker chamber and control (0-) cells were washed four times with cold Ca²⁺-free PBS and frozen as cell pellets at −70° C. for mass spectrometric analysis. All centrifugation steps were performed at 300×g for 5 min. 1×10⁶ BM-MSCs were suspended in 300 μl reaction buffer consisting of αMEM and 0.5% bovine serum albumin (BSA, >=99% pure). The enzymatic reactions were performed in 24-well cell culture plates in a humidified 5% CO₂ atmosphere at +37° for either 2 or 4 hours. The reactions were controlled for attachment to the cell culture dish by suspending the cells every 30 min during the incubations. Control reactions were performed simultaneously with cells in only reaction buffer for 2- or 4 hours. The following enzymatic conditions were tested 1) 50 mU recombinant α2,3-(N)-Sialyltransferase (Calbiochem) and 1 mg CMP-Neu5Ac (donor) 2) 10 mU (⅕) recombinant α2,3-(N)-Sialyltransferase and 1 mg CMP-Neu5Ac 3) 50 mU recombinant α2,3-(N)-Sialyltransferase and 0.2 mg (⅕) CMP-Neu5Ac and 4) 50 mU inactivated (boiled for 5 min and transferred directly to ice) recombinant α2,3-(N)-Sialyltransferase and 1 mg CMP-Neu5Ac. The enzymatic reactions were stopped by adding excess (2 ml) cold Ca²⁺-free PBS or Ca²⁺-free PBS supplemented with 75 mM lactose to the reactions. Cell viability was determined by Trypan blue staining and microscopic analysis in a Bürker chamber. The cells were centrifuged at 300×g for 5 min and washing was repeated additionally 3 times. After the last wash the cells were divided in two and half the cells were pelleted by centrifugation and frozen at −70° C. for further mass spectrometric N-glycan analysis and half the cells were used subsequently in flow cytometric analysis. The MALDI-TOF mass spectrometric analysis was performed for N-glycosidase F liberated N-glycans essentially as described (Hemmoranta H. et al., 2007. Exp. Hematol.).

### Surface N-Glycan Analysis

Some samples were labelled with sulfosuccinimidyl 2-(biotinamido)-ethyl-1,3-dithiopropionate (Sulfo-NHS-SS-Biotin, Pierce) as described by the manufacturer for 30 min at +4° C. The labelling was stopped by adding 500 μl quenching solution and washed twice with excess Tris buffered saline (TBS). The cells were frozen as cell pellets at −70° C.

### Flow Cytometry (FACS)

BM-MSCs were phenotyped by flow cytometric analysis (FACSAria, Becton Dickinson) after the enzymatic reactions. Fluorescein isothiocyanate (FITC), phycoerythrin (PE) or allophycocyanin (APC)-cyanine (Cy)7 conjugated anti-human antibodies against CD90 (Stem Cell Technologies), CD45, CD34, CD14, CD19, CD106, CD73 and HLA-DR (all from BD Biosciences, San Jose, Calif.) were used for direct labelling using 1×10⁵ cells per reaction in Ca²⁺-free PBS supplemented with 1% BSA. Analysis was performed using the FACSDiva software (Beckton Dickinson).

### Results

The viability of the cells did not differ between the different conditions tested, indicating that it was not affected by the enzymatic modification reaction. Also the immunophenotype did not change during the enzymatic modification and the cells were consistently concluded to be strongly positive for CD90 and CD73 and negative (or weakly positive) for CD45, CD34, CD14 and CD19. The enzymatic modifications did not either alter HLA-DR expression of the used BM-MSCs. The results are presented in below Table 8

Cellular Glycan Modification with Sialyltransferase

In the reaction, cell surface terminal N-acetyllactosamine units were sialylated as demonstrated by N-glycan structural analyses as follows. Reaction efficiency and level of terminal LN modification was followed by MALDI-TOF mass spectrometric profiling of the neutral N-glycan fraction, as described in the preceding Experiments. Three glycan signals were used as indicators of the reaction level, namely at m/z 1622 (corresponding to hybrid-type N-glycan Hex6HexNAc3 Na-adduct signal, with documented one terminal LN unit), m/z 1663 (corresponding to complex-type N-glycan Hex5HexNAc4 Na-adduct signal, with documented two terminal LN units), and m/z 2028 (corresponding to complex-type N-glycan Hex6HexNAc5 Na-adduct signal, with documented three terminal LN units). These three indicator signals were good indicators for the overall sialylation level change. In the table below, reaction level is calculated by the equation:

100%−100%*(I₁₆₂₂+I₁₆₆₃+I₂₀₂₈)ₐ/(I₁₆₂₂+I₁₆₆₃+I₂₀₂₈)_{b}

wherein Iₓ is relative proportion of glycan signal x (% of total glycan profile), a indicates signals after enzyme reaction and b control reaction. The disappearance of sialic acid from the molecules indicates the increase of sialylation level.
which corresponds to the proportion (%) of N-glycan signals with terminal LN units that disappeared from the neutral glycan profile during the reaction.

The glycan signal at m/z 1257 (control, corresponding to Na-adduct of Hex5HexNAc2 high-mannose type N-glycan) stayed between 5.7%-6.8% in all conditions, showing that modification was specific.

condition: relative amount of glycan signals (% of total glycan profile):

| | 1622 | 1663 | 2028 | reaction level (%) |
|---|---|---|---|---|
| 0- (cells in culture) | 1.33% | 2.10% | 0.40% | 3% |
| buffer control | 1.54% | 2.02% | 0.40% | 0% |
| inactivated enzyme | 1.34% | 1.94% | 0.48% | 5% |
| 2 h reaction | 0.77% | 0.32% | 0.00% | 72% |
| 4 h reaction | 0.52% | 0.32% | 0.00% | 79% |
| 1:5 donor | 0.89% | 0.54% | 0.00% | 64% |
| 1:5 enzyme | 0.95% | 0.67% | 0.00% | 59% |

The results indicated that:

1) exogenous enzyme was needed for efficient reaction, as indicated by negligible low reaction levels in reaction with heat-inactivated enzyme (5% reaction level) when compared to both original cells (0%, by reference) and buffer control (3%); however low reaction was seen in the inactivated enzyme reaction with supplemented donor substrate CMP-NeuAc (5%),
2) the highest effective reaction level was c. 80% (79%) in the optimized reaction conditions,
3) 2 h reaction time (72%) was nearly as efficient as 4 h reaction (79%), however longer reaction time produced higher reaction level,
4) less amount of either enzyme (1:5 enzyme) or donor substrate (1:5 donor) resulted in less efficient reaction (59% and 64%, respectively), showing that the optimized reaction conditions were critical for efficient removal of cell surface LN units to take place; however, enzyme amount was more critical for reaction efficiency than donor amount above the 1:5 donor concentration.

When the cell culture and reaction media were analyzed by the same method, it was additionally detected that LN group containing glycoprotein components when added to the medium were efficient substrates of the enzymatic modification, therefore competing with the cell surface modification if added to the reaction solution.

The present reaction levels (α2,3-sialyltransferase reaction with MSC) are significantly higher than the reaction level (45%) calculated by the same equation for data in Table 5 (α2,3-sialyltransferase reaction with MNC, see Example 3) and corresponding substantially smaller changes of % units of individual glycans in Table 4.

Contamination with Insect Derived Enzyme and its Removal

m/z 1079, corresponding to sodium adduct ion of Hex3HexNAc2dHex1, a paucimannosidic insect N-glycan/low-mannose type human N-glycan. Below are results from mass spectrometric measurements of the relative amounts of m/z 1079 glycan signal in different reaction/wash conditions.

condition: relative amount of m/z 1079 glycan signal (% of total glycan profile):

| | | Difference to | Relative |
|---|---|---|---|
| | | control % | difference to 4 h |
| buffer control | 0.75% | | |
| 4 h reaction | 1.45% | 0.70 | |
| inactivated enzyme | 1.48% | 0.73 | |
| 1:5 donor | 1.47% | 0.72 | |
| 1:5 enzyme | 1.09% | 0.34 | 49% |
| wash opt A | 0.84% | 0.09 | 12% |

The results indicated that:

1) low level of the m/z 1079 glycan signal were present in the cells before addition of the enzyme due to endogenous low-mannose N-glycans (0.75% relative amount in the “buffer control” condition),
2) all reaction conditions with full amount of enzyme resulted in contamination with insect-derived glycan (c. 1.5% relative amount in the “4 h reaction”, “inactivated enzyme”, and “1:5 donor” conditions),
3) less amount of added enzyme resulted in less contamination (1.1% relative amount in the “1:5 enzyme” condition),
4) 75 mM lactose included in the washing buffers resulted in efficient wash of the insect-derived glycan contamination (0.84% relative amount in the “wash opt A” condition), and
5) incomplete reaction may increase enzyme contamination (1.47% versus 1.45% in the “1:5 donor” and “4 h reaction” conditions, respectively).

### Example 9

### Production a Tag Glyco-Conjugated Enzyme

Mammalian glycosyltransferase (e.g. β4-galactosyltransferase, bovine GalT1) is treated with α-sialidase, and β-galactosidase. Ketone modified Gal is transferred from ketone modified Gal-UDP to the terminal monosaccharide GlcNAc-residue by mutant galactosyltransferase as described in patent application by part of the inventors US2005014718 (included fully as reference) or by Qasba and Ramakrishman and colleagues US2007258986 (included fully as reference) or by using methods described in glycopegylation patenting of Neose (US2004132640, included fully as reference). The ketone is reacted with excess of amino-oxy-biotin (or hydrazide-biotin).

TABLE 1
Detection of N-glycolylneuraminic acid (Neu5Gc) containing sialylated
N-glycans in stem cells and cells differentiated therefrom.

| | | |
|---|---|---|
| Proposed monosaccharide composition | m/z (calculated) | m/z (experimental) |

Human embryonal stem cell line:

| | | |
|---|---|---|
| NeuGcHex3HexNAc3dHex2/NeuAcHex4HexNAc3dHex | 1711.61 | 1711.74 |
| NeuGcHex4HexNAc3dHex/NeuAcHex5HexNAc3 | 1727.60 | 1727.68 |
| NeuGcHex5HexNAc4 | 1946.67 | 1946.7 |
| NeuGcHex5HexNAc4dHex/NeuAcHex6HexNAc4 | 2092.73 | 2092.73 |
| NeuGcNeuAcHex5HexNAc4 | 2237.77 | 2237.7 |
| NeuGc2Hex5HexNAc4 | 2253.76 | 2253.73 |
| NeuGcHex6HexNAc4dHex/NeuAcHex7HexNAc4 | 2254.79 | 2254.73 |
| NeuAc2Hex6HexNAc4/NeuGcNeuAcHex5HexNAc4dHex | 2383.83 | 2383.72 |
| NeuGcHex5HexNAc4dHex3/NeuAcHex6HexNAc4dHex2 | 2384.85 | 2384.72 |
| NeuGcNeuAc2Hex5HexNAc4 | 2528.87 | 2528.77 |
| NeuGc2NeuAcHex5HexNAc4 | 2544.86 | 2544.89 |
| NeuGc2Hex5HexNAc4dHex2/NeuGcNeuAcHex6HexNAc4dHex | 2545.88 | 2545.97 |

Human bone marrow mesenchymal stem cell line:

| | | |
|---|---|---|
| NeuGcHex3HexNAc3dHex2/NeuAcHex4HexNAc3dHex | 1711.61 | 1711.87 |
| NeuGcHex4HexNAc3dHex/NeuAcHex5HexNAc3 | 1727.60 | 1727.87 |
| NeuGcHex5HexNAc3 | 1743.60 | 1743.7 |
| NeuGcHex3HexNAc5 | 1825.65 | 1825.91 |
| NeuGcHex5HexNAc4 | 1946.67 | 1946.92 |
| NeuGcNeuAcHex4HexNAc3dHex/NeuAc2Hex5HexNAc3 | 2018.70 | 2018.88 |
| NeuGcHex5HexNAc4dHex/NeuAcHex6HexNAc4 | 2092.73 | 2093 |
| NeuAcHex7HexNAc4/NeuGcHex6HexNAc4dHex | 2254.79 | 2255.03 |
| NeuAcHex7HexNAc5dHex/NeuGcHex6HexNAc5dHex2 | 2603.92 | 2604.13 |

Cord blood CD 133⁺ cells, sialylated N-glycans:

| | | |
|---|---|---|
| NeuGcHex3HexNAc3 | 1419.49 | 1419.68 |
| NeuGcHex4HexNAc3 | 1581.54 | 1581.74 |
| NeuGcHex3HexNAc3dHex2/NeuAcHex4HexNAc3dHex | 1711.61 | 1711.82 |
| NeuGcHex4HexNAc3dHex/NeuAcHex5HexNAc3 | 1727.60 | 1727.83 |
| NeuGcHex4HexNAc4 | 1784.62 | 1784.85 |
| NeuGcHex4HexNAc3dHex2/NeuAcHex5HexNAc3dHex | 1873.66 | 1873.89 |
| NeuGcHex5HexNAc4 | 1946.67 | 1946.9 |
| NeuGcNeuAcHex4HexNAc3dHex/NeuAc2Hex5HexNAc3 | 2018.70 | 2018.91 |
| NeuGcHex5HexNAc4SO3 | 2026.63 | 2026.82 |
| NeuGcHex5HexNAc4dHex/NeuAcHex6HexNAc4 | 2092.73 | 2092.95 |
| NeuGcNeuAcHex5HexNAc4 | 2237.77 | 2237.97 |
| NeuGcHex5HexNAc4dHex2/NeuAcHex6HexNAc4dHex | 2238.79 | 2238.97 |
| NeuGc2Hex5HexNAc4 | 2253.76 | 2253.97 |
| NeuGcHex6HexNAc4dHex/NeuAcHex7HexNAc4 | 2254.79 | 2254.97 |
| NeuAc2Hex6HexNAc4/NeuGcNeuAcHex5HexNAc4dHex | 2383.83 | 2384.03 |
| NeuGcHex5HexNAc4dHex3/NeuAcHex6HexNAc4dHex2 | 2384.85 | 2385.04 |
| NeuGc2Hex5HexNAc4dHex/NeuGcNeuAcHex6HexNAc4 | 2399.82 | 2400.02 |
| NeuGcNeuAc2Hex5HexNAc4 | 2528.87 | 2529.05 |
| NeuGc2NeuAcHex5HexNAc4 | 2544.86 | 2545.06 |
| NeuGc2Hex5HexNAc4dHex2/NeuGcNeuAcHex6HexNAc4dHex | 2545.88 | 2546.06 |
| NeuGcNeuAcHex6HexNAc5 | 2602.90 | 2603.08 |
| NeuGcHex6HexNAc5dHex2/NeuAcHex7HexNAc5dHex | 2603.92 | 2604.09 |
| NeuGcHex8HexNAc5dHex/NeuAcHex9HexNAc5 | 2781.97 | 2782.18 |
| NeugcNeuAc2Hex6HexNAc5 | 2894.00 | 2894.2 |
| NeuGcNeuAcHex6HexNAc5dHex2/NeuAc2Hex7HexNAc5dHex | 2895.00 | 2895.15 |

TABLE 2
Differential effect of α2,3-sialidase treatment on isolated sialylated
N-glycans from cord blood CD133⁺ and CD133⁻ cells.

| | Proposed monosaccharide | Sialylated N-glycan | | Neutral N-glycan | |
|---|---|---|---|---|---|
| m/z | composition | CD133⁺ | CD133⁻ | CD133⁺ | CD133⁻ |
| 1768 | (NeuAc₁)Hex₄HexNAc₄ | + | + | + | − |
| 2156 | (NeuAc₁)Hex₈HexNAc₂dHex₁/ | + | + | + | − |
| | (NeuAc₁Hex₅HexNAc₄dHex₁SO₃) | | | | |
| 2222 | (NeuAc₁)Hex₅HexNAc₄dHex₂ | + | + | + | − |
| 2238 | (NeuAc₁Hex₆HexNAc₄dHex₁/ | + | + | + | − |
| | (NeuGc₁)Hex₅HexNAc₄dHex₂ | | | | |
| 2254 | (NeuAc₁)Hex₇HexNAc₄/ | + | + | + | − |
| | (NeuGc₁)Hex₆HexNAc₄dHex₁ | | | | |
| 2368 | (NeuAc₁)Hex₅HexNAc₄dHex₃ | + | + | + | − |
| 2447 | (NeuAc₂)Hex₈HexNAc₂dHex₁/ | + | + | + | − |
| | (NeuAc₂Hex₅HexNAc₄dHex₁SO₃) | | | | |
| 2448 | (NeuAc₁)Hex₈HexNAc₂dHex₃/ | + | + | + | − |
| | (NeuAc₁Hex₅HexNAc₄dHex₃SO₃) | | | | |
| 2513 | (NeuAc₂)Hex₅HexNAc₄dHex₂ | + | + | + | − |
| 2733 | (NeuAc₁)Hex₆HexNAc₅dHex₃ | + | + | + | − |
| 2953 | (NeuAc₁)Hex7HexNAc₆dHex₂ | + | + | + | − |

The neutral N-glycan columns show that neutral N-glycans corresponding to the listed sialylated N-glycans appear in analysis of CD133⁺ cell N-glycans but not CD133⁻ cell N-glycans. Proposed glycan compositions outside parenthesis are visible in the neutral N-glycan fraction after α2,3-sialidase digestion of CD133⁺ cell sialylated N-glycans.

TABLE 3
Cord blood mononuclear cell sialylated N-glycan signals.

| Proposed monosaccharide composition | m/z (calculated) | |
|---|---|---|
| NeuAcHex3HexNAc3dHex | 1549.55 | 1549 |
| NeuAcHex4HexNAc3 | 1565.55 | 1565 |
| NeuAc2Hex3HexNAc2dHex | 1637.57 | 1637 |
| NeuAc2Hex2HexNAc3dHex | 1678.60 | 1678 |
| NeuAcHex4HexNAc3dHex | 1711.61 | 1711 |
| NeuAcHex5HexNAc3 | 1727.60 | 1727 |
| NeuAcHex3HexNAc4dHex | 1752.63 | 1752 |
| NeuAcHex4HexNAc4 | 1768.57 | 1768 |
| NeuAcHex4HexNAc3dHexSO3 | 1791.56 | 1791 |
| NeuAc2Hex3HexNAc3dHex | 1840.65 | 1840 |
| NeuAcHex4HexNAc3dHex2 | 1857.66 | 1857 |
| Hex5HexNAc4dHexSO3 | 1865.60 | 1865 |
| NeuAcHex5HexNAc3dHex | 1873.66 | 1873 |
| NeuAcHex6HexNAc3 | 1889.65 | 1889 |
| NeuAcHex3HexNAc4dHex2 | 1898.69 | 1898 |
| NeuAcHex4HexNAc4dHex | 1914.68 | 1914 |
| NeuAcHex5HexNAc4 | 1930.68 | 1930 |
| NeuAc2Hex4HexNAc3dHex/Hex8HexNAc3SO3 | 2002.70 | 2002 |
| NeuAc2Hex5HexNAc3 | 2018.70 | 2018 |
| NeuAcHex6HexNAc3dHex | 2035.71 | 2035 |
| NeuAcHex7HexNAc3 | 2051.71 | 2051 |
| Hex4HexNAc5dHex2SO3 | 2052.68 | 2052 |
| NeuAc2Hex4HexNAc4 | 2059.72 | 2059 |
| NeuAcHex4HexNAc4dHex2 | 2060.74 | 2060 |
| NeuAcHex5HexNAc4dHex | 2076.74 | 2076 |
| NeuAcHex6HexNAc4 | 2092.73 | 2092 |
| NeuAcHex4HexNAc5dHex | 2117.76 | 2117 |
| NeuAcHex5HexNAc5 | 2133.76 | 2133 |
| NeuAcHex8HexNAc2dHex/NeuAcHex5HexNAc4dHexSO3 | 2156.74/2156.69 | 2156 |
| NeuAc2Hex5HexNAc4 | 2221.78 | 2221 |
| NeuAcHex5HexNAc4dHex2 | 2222.80 | 2222 |
| Hex6HexNAc5dHexSO3 | 2230.73 | 2230 |
| NeuAcHex6HexNAc4dHex/NeuGcHex5HexNAc4dHex2 | 2238.79 | 2238 |
| NeuAcHex7HexNAc4/NeuGcHex6HexNAc4dHex | 2254.79 | 2254 |
| NeuAcHex5HexNAc5dHex | 2279.82 | 2279 |
| NeuAc2Hex4HexNAc3dHex3 | 2294.82 | 2294 |
| NeuAcHex6HexNAc5 | 2295.81 | 2295 |
| NeuAc2Hex5HexNAc4dHex | 2367.83 | 2367 |
| NeuAcHex5HexNAc4dHex3 | 2368.85 | 2368 |
| NeuAc2Hex6HexNAc4 | 2383.83 | 2383 |
| NeuAcHex6HexNAc4dHex2 | 2384.85 | 2384 |
| NeuAc2Hex5HexNAc3dHexSO3 | 2390.77 | 2390 |
| NeuAc2Hex3HexNAc5dHex2 | 2392.86 | 2392 |
| NeuAcHex5HexNAc5dHex2 | 2425.87 | 2425 |
| NeuAcHex6HexNAc5dHex | 2441.87 | 2441 |
| NeuAc2Hex8HexNAc2dHex/NeuAc2Hex5HexNAc4dHexSO3 | 2447.83/2447.79 | 2447 |
| NeuAcHex7HexNAc5 | 2457.86 | 2457 |
| NeuAc2Hex5HexNAc4dHex2 | 2513.89 | 2513 |
| NeuAcHex6HexNAc5dHexSO3 | 2521.83 | 2521 |
| NeuAcHex6HexNAc4dHex3 | 2530.91 | 2530 |
| NeuAc3Hex4HexNAc5 | 2553.90 | 2553 |
| NeuAc2Hex5HexNAc5dHex | 2570.91 | 2570 |
| NeuAcHex5HexNAc5dHex3 | 2571.93 | 2571 |
| NeuAc2Hex6HexNAc5 | 2586.91 | 2586 |
| NeuAcHex6HexNAc5dHex2 | 2587.93 | 2587 |
| Hex7HexNAc6dHexSO3 | 2595.86 | 2595 |
| NeuAcHex7HexNAc5dHex | 2603.92 | 2603 |
| NeuAcHex6HexNAc6dHex | 2644.95 | 2644 |
| NeuAcHex7HexNAc6 | 2660.94 | 2660 |
| NeuAc2Hex4HexNAc5dHex2(SO3)2 | 2714.83 | 2714 |
| NeuAc2Hex6HexNAc5dHex | 2732.97 | 2732 |
| NeuAcHex6HexNAc5dHex3 | 2733.99 | 2733 |
| NeuAcHex7HexNAc6dHex | 2807.00 | 2807 |
| NeuAcHex6HexNAc5dHex3SO3 | 2813.94 | 2813 |
| NeuAc3Hex6HexNAc5 | 2878.00 | 2878 |
| NeuAc2Hex6HexNAc5dHex2 | 2879.02 | 2879 |
| NeuAcHex6HexNAc5dHex4 | 2880.04 | 2880 |
| NeuAc2Hex5HexNAc6dHex2 | 2920.05 | 2920 |
| NeuAc2Hex7HexNAc6 | 2952.04 | 2952 |
| NeuAcHex7HexNAc6dHex2 | 2953.06 | 2953 |
| NeuAcHex7HexNac7dHex | 3010.08 | 3010 |
| NeuAc3Hex6HexNAc5dHex | 3024.06 | 3024 |
| NeuAc2Hex6HexNAc5dHex3 | 3025.09 | 3025 |
| NeuAcHex8HexNAc7 | 3026.08 | 3026 |
| NeuAc2Hex7HexNAc6dHex | 3098.10 | 3098 |
| NeuAcHex7HexNAc6dHex3 | 3099.12 | 3099 |
| NeuAc2Hex6HexNAc5dHex4 | 3171.14 | 3171 |
| NeuAcHex8HexNAc7dHex | 3172.13 | 3172 |

The m/z values refer to monoisotopic masses of [M-H]⁻ ions.

TABLE 4
Mass spectrometric analysis results of sialylated N-glycans with mono-
saccharide compositions NeuAc₁₋₂Hex₅HexNAc₄dHex₀₋₃ in sequential
enzymatic modification steps of human cord blood mononuclear cells.

| Proposed monosaccharide | calc m/z | | | α2,3SAT + |
|---|---|---|---|---|
| composition | [M-H]⁻ | MNC | α2,3SAT | α1,3FucT |
| NeuAcHex5HexNAc4 | 1930.68 | 24.64 | 12.80 | 13.04 |
| NeuAcHex5HexNAc4dHex | 2076.74 | 39.37 | 30.11 | 29.40 |
| NeuAcHex5HexNAc4dHex2 | 2222.8 | 4.51 | 8.60 | 6.83 |
| NeuAcHex5HexNAc4dHex3 | 2368.85 | 3.77 | 6.34 | 6.45 |
| NeuAc2Hex5HexNAc4 | 2221.78 | 13.20 | 12.86 | 17.63 |
| NeuAc2Hex5HexNAc4dHex | 2367.83 | 14.04 | 29.28 | 20.71 |
| NeuAc2Hex5HexNAc4dHex2 | 2513.89 | 0.47 | n.d. | 5.94 |

The columns show relative glycan signal intensities (% of the tabled signals) before the modification reactions (MNC), after α2,3-sialyltransferase reaction (α2,3SAT), and after sequential α2,3-sialyltransferase and α1,3-fucosyltransferase reactions (α2,3SAT + α1,3FucT). The sum of the glycan signal intensities in each column has been normalized to 100% for clarity.

TABLE 5
Mass spectrometric analysis results of selected neutral N-glycans in
enzymatic modification steps of human cord blood mononuclear cells.

| Proposed monosaccharide | calc m/z | | | | | α2,3SAT + |
|---|---|---|---|---|---|---|
| composition | [M + H]⁺ | MNC | SA'ase | α2,3SAT | α1,3FucT | α1,3FucT |
| Hex5HexNAc2 | 1257.42 | 11.94 | 14.11 | 14.16 | 13.54 | 9.75 |
| Hex3HexNAc4dHex | 1485.53 | 0.76 | 0.63 | 0.78 | 0.90 | 0.78 |
| Hex6HexNAc3 | 1622.56 | 0.61 | 1.99 | 0.62 | 0.51 | 0.40 |
| Hex5HexNAc4 | 1663.58 | 0.44 | 4.81 | 0.00 | 0.06 | 0.03 |
| Hex5HexNac4dHex | 1809.64 | 0.19 | 1.43 | 0.00 | 0.25 | 0.00 |
| Hex5HexNac4dHex2 | 1955.7 | 0.13 | 0.22 | 0.00 | 0.22 | 0.00 |
| Hex6HexNAc5 | 2028.71 | 0.07 | 1.14 | 0.00 | 0.00 | 0.00 |
| Hex5HexNAc4dHex3 | 2101.76 | 0.12 | 0.09 | 0.00 | 0.22 | 0.00 |
| Hex6HexNAc5dHex | 2174.77 | 0.00 | 0.51 | 0.00 | 0.14 | 0.00 |
| Hex6HexNAc5dHex2 | 2320.83 | 0.00 | 0.00 | 0.00 | 0.08 | 0.00 |

The columns show relative glycan signal intensities (% of the total glycan signals) before the modification reactions (MNC), after broad-range sialidase reaction (SA'se), after α2,3-sialyltransferase reaction (α2,3SAT), after α1,3-fucosyltransferase reaction (α1,3FucT), and after sequential α2,3-sialyltransferase and α1,3-fucosyltransferase reactions (α2,3SAT + α1,3FucT).

TABLE 6
Proposed monosaccharide compositions for NeuGc-containing or O-acetylated
sialic acid containing glycans and their calculated isotopic masses

| Proposed structures | m/z isotopic |
|---|---|
| NeuGcHex3HexNAc3 | 1419.48964 |
| NeuAcHex3HexNAc3Ac | 1445.50529 |
| NeuGcHex4HexNAc3 | 1581.54246 |
| NeuAcHex4HexNAc3dHex/NeuGcHex3HexNAc3dHex2 | 1711.60545 |
| NeuGc2Hex3HexNAc3 | 1726.57996 |
| NeuAcHex5HexNAc3/NeuGcHex4HexNAc3dHex | 1727.60036 |
| NeuGcHex5HexNAc3 | 1743.59528 |
| NeuGcHex4HexNac4 | 1784.62183 |
| NeuAcHex5HexNAc3dHex/NeuGcHex4HexNAc3dHex2 | 1873.65827 |
| NeuGcHex5HexNAc4 | 1946.67465 |
| NeuAcHex5HexNAc4Ac | 1972.6903 |
| NeuAc2Hex5HexNAc3/NeuGcNeuAcHex4HexNAx3dHex | 2018.69578 |
| NeuGcHex5HexNAc4SP | 2026.63146 |
| NeuGc2Hex4HexNAc4 | 2091.71215 |
| NeuAcHex6HexNAc4/NeuGcHex5HexNAc4dHex | 2092.73255 |
| NeuAcHex9HexNAc2/NeuAcHex6HexNAc4SP/NeuGcHex5HexNAc4dHexSP | 2172.732/2172.689 |
| NeuAc2Hex6HexNAc3/NeuGcHex4HexNac3dHex2 | 2180.7486 |
| NeuGcNeuAc2Hex2HexNAc4dHex | 2188.76491 |
| NeuGcNeuAcHex6HexNAc3 | 2196.74351 |
| NeuGcNeuAcHex5Hexnac4 | 2237.77006 |
| NeuAcHex6HexNAc4dHex/NeuGcHex5HexNAc4dHex2 | 2238.79046 |
| NeuGc2Hex5Hexnac4 | 2253.76497 |
| NeuAcHex7HexNAc4/NeuGcHex6HexNAc4dHex | 2254.78537 |
| NeuAc2Hex5HexNAc4Ac | 2263.78571 |
| NeuAc2Hex5HexNAc4Ac2 | 2305.79627 |
| NeuAc2Hex5HexNAc3dHex2/NeuGcNeuAcHex4HexNAc3dHex3 | 2310.81159 |
| NeuAcHex5HexNAc3dHex4NeuGcHex6HexNAc5 | 2311.83199 |
| Hex6HexNAc4dHex3SP/NeuGcNeuAcHex3HexNAc6 | 2319.76768 |
| NeuGcHex3HexNAc6dHex2 | 2320.84355 |
| NeuAcHex5HexNAc5dHexAc | 2321.82757 |
| NeuGcHex6HexNAc4dHexSP | 2334.74219 |
| NeuGcNeuAc2Hex4HexNAc4 | 2366.81265 |
| NeuAc2Hex6HexNAc4/NeuGcNeuAcHex5HexNAc4dHex | 2383.82797 |
| NeuAcHex6HexNAc4dHex2/NeuGcHex5HexNAc4dHex3 | 2384.84837 |
| NeuAc2Hex5HexNAc4Ac4 | 2389.8174 |
| NeuGc2Hex5HexNAc4dHex | 2399.82288 |
| NeuAc2Hex5HexNAc4dHexAc | 2409.84361 |
| NeuAc2Hex5HexNAc4dHexAc2 | 2451.85418 |
| NeuAcHex5HexNAc5dHex2Ac | 2467.88548 |
| NeuAcHex6HexNAc5Ac | 2483.88039 |
| NeuGcNeuAc2Hex5HexNAc4 | 2528.86547 |
| NeuAc2Hex6HexNAc4dHex/NeuGcNeuAcHex5HexNAc4dHex2 | 2529.88587 |
| NeuGc2NeuAcHex5HexNAc4 | 2544.86039 |
| NeuGc2Hex5Hexnac4dHex2/NeuGcNeuAcHex6HexNAc4dHex | 2545.88079 |
| NeuGc3Hex5HexNAc4 | 2560.8553 |
| NeuGcNeuAcHex6HexNAc5 | 2602.90225 |
| NeuAcHex7HexNAc5dHex/NeuGcHex6HexNAc5dHex2 | 2603.92265 |
| NeuGc2Hex6HexNac5 | 2618.89716 |
| NeuGcHex7HexNAc5dHex | 2619.91756 |
| NeuGcHex8HexNAc5 | 2635.91248 |
| NeuGcNeuAc2Hex5HexNAc4dHex | 2674.92338 |
| NeuGcHex6HexNAc5dHex2SP | 2683.87947 |
| NeuAc2Hex6HexNAc3dHex4/NeuGc2Hex6HexNAc5dHex | 2764.980/764.955 |
| NeuAcHex9HexNAc5/NeuGcHex8HexNAc5dHex | 2781.97038 |
| NeuGcNeuAc2Hex6HexNAc5 | 2893.99766 |
| NeuAc2Hex7HexNAc5dHex/NeuGcNeuAcHex6HexNAc5dHex2 | 2895.01806 |
| NeuGc2Hex6HexNAc5dHex2 | 2911.01298 |
| NeuGc3Hex6HexNAc5 | 2925.98749 |
| NeuGcNeuAc2Hex5HexNAc6 | 2935.02421 |
| NeuGcNeuAcHex7HexNAc6dHex2 | 3260.15025 |
| NeuGcHex7HexNAc6dHex4 | 3261.17065 |

TABLE 7
Effect of fucosylation, sialylation and neuraminidase on viability and differentiation of MNC.

| Condition | Buffer | Cell number | Incubation time | Viability (%) | Viability (%) |
|---|---|---|---|---|---|
| Original cell suspension | — | — | — | 92.7 | 86.5 |
| Control (Buffer) | HBSS - 1% HSA - 10 mM | 10 × 10⁶ | 60 min | 95.7 | 95.2 |
| Fucosyltransferase treat | HBSS - 1% HSA - 10 mM | 10 × 10⁶ | 60 min | 97.1 | 92.5 |
| Sialylation treatment | HBSS - 1% HSA - 10 mM | 10 × 10⁶ | 60 min | 96.3 | 92.3 |
| Neuraminidase treatment | HBSS - 1% HSA - 10 mM | 10 × 10⁶ | 60 min | 96.9 | 95.7 |

| | EXP 1 | EXP 1 |
|---|---|---|
| Condition | CFU tot mean | CFU tot mean |
| Control (Buffer) | 82 | 137.5 |
| Fucosylation | 58.5 | 138 |
| Sialylation | 75.5 | 110 |
| Neuraminaidase | 109.5 | 196.5 |

TABLE 8
Viability and immunophenotype of BM-MSCs after
α2,3-Sialyltransferase enzymatic modifications

| | Viability | FACS: % positive | | | | |
|---|---|---|---|---|---|---|
| | % | CD90 | CD45/CD34/CD14/CD19 | HLA-DR | CD106 | CD73 |
| Controls 2 h reaction buffer | 50 | 99.4 | 4 | 41.1 | 2.3 | 91.1 |
| 4 h reaction buffer | 60 | 99 | 3.5 | 38.6 | 2.7 | 83.3 |
| Inactivated enzyme | 50 | 98.3 | 7.1 | 52.8 | 3.2 | 85.5 |
| 2 h 50 mU reaction | 50 | 99.4 | 6.2 | 44.1 | 3.7 | 94.7 |
| 4 h 50 mU reaction | 50 | 99 | 2.9 | 43.3 | 3.5 | 87.1 |
| 1/5 donor | 40 | 98.7 | 3.9 | 43.1 | 2.8 | 76.1 |
| 1/5 enzyme | 60 | 99.1 | 3.6 | 42.4 | 4.1 | 88.8 |
| PBS + 75 mM lactose wash | 60 | 99.4 | 5.7 | 40.1 | 4.2 | 95.6 |

1-59. (canceled)

60. A method of altering the sialylation or fucosylation of embryonal stem cells, cord blood cells, hematopoietic stem cells or mesenchymal stem cells, comprising the step(s) of
a) sialylating the cells by the use of CMP-sialic acid and specific sialyltransferase enzyme and/or
b) desialylating the cells by sialidase enzyme and/or
c) fucosylating the cells by the use of GDP-Fuc and specific fucosyltransferase enzyme;
1) wherein the enzyme is tagged chemically to amine, thiol or oxidized glycan and/or
2) the enzyme is removed using an inhibitor binding to substrate binding site of the enzyme.

61. The method according to claim 60, wherein the enzyme comprises a structure according to
B-(G-)ₘR1-R2-(S1-)ₙT Formula CONJ
wherein B is the enzyme, G is glycan (when the enzyme is glycan conjugated),
R1 and R2 are chemoselective ligation groups, T is tag, preferably biotin; S1 is optional spacer group, preferably C₁-C₁₀ alkyls, m, and n are integers being either 0 or 1, independently.

62. The method according to claim 60, wherein the enzyme is tagged by binding covalently to enzyme surface a tagging group selected from a group antigen, biotin, his-tag, chemical tag, fluoroalkane or biotin, preferably biotin of fluoroalkane.

63. The method according to claim 60, wherein the enzyme is glycan tagged.

64. The method according to claim 60, wherein the enzyme is selected form the group consisting of a sialyltransferase or fucosyltransferase which reacts with N-acetyllactosamines, FTIII, FTIV, FTV, FTVI, FTVII and FTIX, ST3GalIII ST3GalIV and ST6GalI, or sialyltransferase reacting with O-glycan core I such as ST3 GalI or ST3GalII and ST3GalIV

65. The method according to claim 60, wherein the tagged enzyme form a complex of tagged enzyme with solid phase according to Formula:
B-(G-)ₘR1-R2-(S1-)ₙT-L-(S2)ₛ-SOL,
wherein B is the enzyme, SOL is solid phase or affinity matrix or polymer or other matrix useful for removal of the enzyme, G is glycan (when the enzyme is glycan conjugated),
R1 and R2 are chemoselective ligation groups, T is tag, preferably biotin, L is specifically binding ligand for the tag; S1 and S2 are optional spacer groups, preferably C₁-C₁₀ alkyls, m, n, and s are integers being either 0 or 1, independently and linkage between T-L can be non-covalent high affinity binding.

66. The method according to claim 60, wherein the tagged modification enzyme is immobilized to a matrix and removed by separating the matrix from the cells.

67. The method according to claim 60, wherein the method involves a step of sialylation with specific α3- and/or α6-linked sialic acids by incubating cells with CMP-sialic acid and sialyltransferase enzyme and/or a step of fucosylation with specific α3- and/or α4-linked fucose by incubating cells with GDP-Fuc acid and fucosyltransferase enzyme.

68. The method according to claim 60, for removing a glycosyltransferase or sialidase modification enzyme from modified cells involving a step of incubation of the cells with an inhibitor or substrate of the enzyme.

69. The method according to claim 60, wherein the inhibitor is competitive monosaccharide glycoside or oligosaccharide inhibitor.

70. The method according to claim 60, wherein the enzyme is sialidase and inhibitor is selected from the group consisting of: competitive low activity inhibitors such as sialic acid, and modified or low cost competing substrates such as NeuAc□OMe, NeuNAc□OEt, sialyl-Lactoses or polysialic acid; higher activity inhibitors such as NeuAc2en or higher activity inhibitors specific for limited number of enzymes or influenza virus neuraminidase inhibitors: Tamiflu (oseltamivir, Roche) or Zanamivir (GSK).

71. The method according to claim 60, wherein the inhibitor oligosaccharide for sialyltransferase inhibition includes sequences including oligosaccharides and reducing end conjugates of Galβ4Glc, Galβ4GlcNAc, Galβ3GlcNAc, Galβ3GalNAc.

72. The method according to claim 60, wherein the inhibitor is a sialidase (neuraminidase) inhibitor, or sialyltransferase inhibitor, or fucosyltransferase inhibitor, preferably lactose, and the method is used together with specifically chemically amine, thiol or glycan tagged sialidase, sialyltransferase or fucosyltransferase enzyme.

73. A cell population prepared or derived from isolated cord blood cells, mesenchymal stem cells, hematopoietic stem cells or embryonal stem cells, wherein the cell population comprises in vitro enzymatically altered sialylation and/or fucosylation, obtainable by
a) sialylating the cells by the use of CMP-sialic acid and specific sialyltransferase enzyme and/or
b) desialylating the cells by sialidase enzyme and/or
c) fucosylating the cells by the use of GDP-Fuc and specific fucosyltransferase enzyme; wherein
1) the enzyme is tagged chemically to amine, thiol or oxidized glycan and
2) the enzyme is removed using an inhibitor binding to substrate binding site of the enzyme.

74. The cell population according to claim 73 wherein the increased sialylated and/or fucosylated structures on the cell surfaces are used for targeting said cells to specific tissues.

75. Mass spectrometric analysis for the presence of the structures on modified cells described in claim 73 comprising releasing of glycans, purification of the glycan fraction, measuring molecular masses; optionally modifying part of glycans by specific sialidase enzymes and analysing the modified glycans; and assigning/fitting the molecular masses of glycans to said specific structures, preferably wherein the sialyation level and optionally the presence or absence of NeuGc is analysed by indicative glycan signals, using rounded exact mass numbers as glycan names, at m/z 1946, m/z 2237, and m/z 2253 or corresponding and additional signal assigned to NeuGc-structures listed in Table 1 and/or Table 6, with optional provision that when the mass number corresponds also to alternative structures the presence of NeuGc is further verified by other data, preferably mass spectrometric or labelling data.

76. A cell population obtained from isolated human stem cells or cord blood cells so that said cells are contacted in vitro with an enzyme altering sialylation of the cells, wherein when the said cells are sialylated or desialylated and optionally the modification enzyme is removed using an inhibitor or the enzyme is tagged, the amount of NeuNAc is increased or decreased at least by 15% units, wherein the said enzymatic modification is used to increase the CFU amount of the said cells,

77. A method involving a step of contacting cord blood cells in vitro with an enzyme altering sialylation and/or fucosylation of the cells to increase CFU in cord blood cell populations.

78. A sialyltransferase or fucosyltransferase or sialidase cell modification enzyme, wherein the transferase or enzyme is covalently tagged to amine or thiol group and/or glycan tagged.

79. The transferase or enzyme according to claim 78, wherein the transferase or enzyme comprises a structure according to
B-(G-)ₘR1-R2-(S1-)ₙT Formula CONJ
wherein B is the transferase or enzyme, G is glycan (when the transferase or enzyme is glycan conjugated),
R1 and R2 are chemoselective ligation groups, T is tag, preferably biotin; S1 is optional spacer group, preferably C₁-C₁₀ alkyls, m, and n are integers being either 0 or 1, independently.
